(19) United States
(12) Patent Application Publication
(10) Pub. No.: US 2023/0374458 A1
(43) Pub. Date: Nov. 23, 2023

(54) COMPOSITIONS AND METHODS FOR GENERATING HUMAN YOLK SAC-LIKE HEMATOPOIETIC CELLS

(71) Applicant: UNIVERSITY HEALTH NETWORK, Toronto (CA)

(72) Inventors: Gordon Keller, Toronto (CA); Michael Atkins, Toronto (CA)

(21) Appl. No.: 18/023,289

(22) PCT Filed: Aug. 25, 2021
(86) PCT No.: PCT/CA2021/051181

§ 371 Date: Feb. 24, 2023

Related U.S. Application Data
(60) Provisional application No. 63/069,904, filed on Aug. 25, 2020.

(51) Int. Cl. C12N 5/0789

(52) U.S. Cl. CPC C12N 5/0647; C12N 2506/45; C12N 2501/115; C12N 2501/155; C12N 2501/16; C12N 2501/724; C12N 2501/42

(57) ABSTRACT

Provided are methods for making yolk sac like hematopoietic progenitors by specifying a KDR+CD235a/b+ mesoderm cells capable of giving rise to T lymphoid lineage cells or cells differentiated therefrom. The method involves contacting pluripotent stem cells (PSCs) with a mesoderm specifying culture composition comprising a BMPR1/R2 agonist, an FGF receptor agonist and an activin receptor agonist to produce a KDR+CD235a/b+ mesoderm cells; and optionally isolating the KDR+CD235a/b+ mesoderm cells.

### RELATED APPLICATIONS

This Patent Cooperation Treaty application claims the benefit of priority of U.S. Provisional Application 63/069,904 filed Aug. 25, 2020, which is incorporated herein in its entirety.

### FIELD

Provided herein are methods and compositions for making and using human yolk sac-like hematopoietic cells from pluripotent stem cells including embryonic stem cells and induced pluripotent stem cells.

### INCORPORATION OF SEQUENCE LISTING

A computer readable form of the Sequence Listing “P62842PC00_ST25” (5,017 bytes) created on Aug. 25, 2021, is herein incorporated by reference.

### INTRODUCTION

Embryonic hematopoiesis in the mouse consists of distinct programs that differ in their lineage potential and spatiotemporal organization. Prior to the generation of hematopoietic stem cells (HSCs), the yolk sac initiates three programs, known as the primitive, erythro-myeloid progenitor (EMP) and lymphoid-primed multipotent progenitor (LMP) programs. While these programs do not contribute to long-term, multilineage hematopoiesis, their innate immune cell progeny seed and persist in the tissues throughout development and adult life. These populations execute organ-specific functions to maintain homeostasis.

Based on studies in model organisms, the yolk sac gives rise to populations with a broad range of lineage potentials, many of which show differences from those produced from the definitive program. Some of these differences reflect the unique needs of the developing embryo, such as its demand for oxygen, while other differences indicate that these cells function beyond early stages of embryogenesis. Lineage tracing and transplantation studies have indeed shown that the yolk sac is a source of specialized tissue-resident immune cells that seed different organs where they persist and function throughout life (Epelman et al., 2014; Gentek et al., 2018a; Gentek et al., 2018b; Ginhoux et al., 2010; Gomez Perdiguero et al., 2015; Hashimoto et al., 2013; Li et al., 2018; Schulz et al., 2012). Many of the adult tissues have a balance between the yolk sac and HSC-derived cells. Although the complete function of these cells is not fully understood, studies using model organisms have shown that these yolk sac-derived cells play a pivotal role in organ regeneration (Aurora et al., 2014; Dick et al., 2019; Lavine et al., 2014).

Hematopoiesis in the mouse is initiated in the yolk sac by distinct programs that collectively produce a broad range of lineages, independent of hematopoietic stem cells (HSCs). The primitive program, the first to develop, has limited potential and gives rise to a transient population of progenitors on embryonic day (E) 7.0, the majority of which are committed to the erythroid lineage. In addition to erythrocytes, primitive hematopoiesis also generates macrophages and megakaryocytes. Following the onset of primitive hematopoiesis, a second program, the erythro-myeloid progenitor (EMP) program emerges at E8.25 and generates an expanded set of lineages that includes granulocytes and mast cells, in addition to the erythroid, macrophage and megakaryocyte lineages. EMPs can be identified and isolated based on the co-expression of CD41, Kit, CD16/32 (FcγRII/III), CD34 and CD45, markers not expressed on primitive hematopoietic progenitors (McGrath et al., 2015). Clonal analyses have demonstrated that EMP hematopoiesis contains CD41+ Kit+CD16/32+ multipotent progenitors that are able to generate the spectrum of lineages produced at this stage (McGrath et al., 2015). Recently it has been demonstrated that this population can also give rise to NK cells (Dege et al., 2020). Although the primitive and EMP programs share common lineages, there are differences in the some of the end stage cells generated. For example, primitive erythrocytes are larger than their EMP counterparts and display a globin expression pattern characterized by the predominance of the embryonic εγ and β H1 globins ( ). In contrast, EMP-derived erythrocytes predominantly express the adult form of β globin, β major along with low level of β H1 globin (McGrath et al., 2015; McGrath et al., 2011; Palis et al., 1999; Wong et al., 1986).

Progenitors with T lymphoid potential have been detected in the yolk sac as early as E9.0 by culture in fetal thymic organs or with OP9 stromal cells (Huang and Auerbach, 1993; Yoshimoto et al., 2012). Stromal cell-based cultures have identified B cell progenitors at the same stage of development (Yoshimoto et al., 2011). Despite the presence of EMPs and lymphoid progenitors in the E9.0 yolk sac, a clonal relationship between these lineages has not been established.

Yolk sac hematopoiesis was long thought to function solely to support the developing embryo prior to the generation of HSCs by the definitive program. Lineage tracing experiments in the mouse have demonstrated that populations of tissue-resident macrophages in the adult, including microglia, Kupffer cells and alveolar macrophages develop from HSC-independent yolk sac-derived progenitors (Ginhoux et al., 2010; Gomez Perdiguero et al., 2015; Schulz et al., 2012). These macrophage progenitors seed the developing organs and generate the tissue-resident populations that maintain themselves throughout adult life (Mass et al., 2016). More recent tracing studies have shown that the contribution of the yolk sac hematopoietic programs to the adult includes subpopulations of mast cells (Gentek et al., 2018a; Li et al., 2018) and T cells (Gentek et al., 2018b). Given the limited access to human tissue at early stages of development, the structure of the human embryonic hematopoietic system is far less well understood than that of the mouse. Studies of the human yolk sac at 5 weeks have identified large, nucleated erythrocytes that express embryonic (HBE; ε) globin indicative of human primitive hematopoiesis. Analyses of colony-forming progenitor potential of the yolk sac and liver at this stage demonstrated the presence of granulocyte and erythroid progenitors. The cells within these erythroid colonies expressed a combination of the embryonic (HBE; ε) and fetal (HBG1/2; Aγ and Gγ) globins suggesting that they originate from the human equivalent of EMP-derived progenitors. Similar globin expression patterns were detected in erythroblasts present in the liver at 6 weeks (Migliaccio et al., 1986; Peschle et al., 1985; Peschle et al., 1984). The detection of rare multipotent progenitors with erythroid, macrophage and granulocyte potential in the yolk sac at Carnegie Stage 12 (approximately day 30) (Bian et al., 2020) has provided further evidence of an EMP program in the human. There are no detailed analyses of lymphoid progenitors in the human yolk sac. Studies on the developing liver and thymus have shown that a subset of γδ T cells, characterized by the expression of the Vδ2 gene segment, are present at five weeks and are a dominant T lymphoid population prior to 12 weeks (Haynes and Heinly, 1995; McVay and Carding, 1996). The proportion of Vδ2+ T cells decrease in these tissues beyond this time and they are supplanted by a second wave made up of Vδ1+ T cells in the fetal blood by 40 weeks (Dimova et al., 2015).

Owing to the scarcity of human yolk sac, efforts to characterize the earliest stages of human embryonic hematopoiesis, such as the relationship of the different hematopoietic programs and the pathways that regulate their specification have turned to the human pluripotent stem cell (hPSC) system to model these developmental steps (Ditadi et al., 2017).

Given the transient nature of their progenitors and their emergence at early stages of development, the identification and study of these programs has not been possible in the human embryo.

Methods and compositions for generating blood lineage progenitor cells are desirable.

### SUMMARY

It is demonstrated herein that the concurrent addition of BMP4, FGF2 and Activin A promote the generation of cultures that are highly enriched in KDR+CD235a/b₊ mesoderm at day 4 of human pluripotent stem cell (hPSC) differentiation that displays the capacity to generate the blood cell lineages indicative of human yolk sac primitive and EMP/lymphoid hematopoiesis. Within 24 hours, the mesoderm gives rise to hemogenic endothelial cells (HECs) on day 5 of differentiation that undergo NOTCH-dependent endothelial-to-hematopoietic transition (EHT) to generate the hematopoietic progenitors of the primitive program. It is demonstrated that the primitive program gives rise to primitive erythroid, mast cells and macrophage lineages. Coincident with the emergence of the primitive hematopoietic progenitors on day 6 of differentiation, it is demonstrated that a second population of HECs give rise to multipotent hematopoietic progenitors that can be distinguished from their primitive counterpart based on the expression of CD45. Multipotent hematopoietic progenitor are enriched in a population defined by CD34⁺CD45⁺CD90⁺CD7⁻. The multipotent progenitor program gives rise to the erythroid, mast cell, macrophage, NK cell and T lymphoid lineages.

Accordingly an aspect includes a method of producing a KDR+CD235a/b−/+ mesoderm cells capable of giving rise to T lymphoid lineage cells or cells differentiated therefrom, the method comprising:
contacting pluripotent stem cells (PSCs) with a PSC culture composition comprising a BMP receptor agonist (BMPRA) and optionally a ROCK inhibitor (Ri) to produce a BMPRA-Ri population of cells;
contacting the BMPRA-Ri population of cells with a mesoderm specifying culture composition comprising a BMPR1/R2 agonist, an FGF receptor agonist and an activin receptor agonist to produce KDR+CD235a/b−/+ mesoderm cells.

As demonstrated KDR+ mesoderm specified as described herein, including CD235a/b+ as well as CD235a/b− can provide yolk sac blood cell lineages.

In an embodiment, the PSCs are contacted with the mesoderm specifying culture composition for about 3 days, at least 3 days or up to 3 days.

In an embodiment, the pluripotent stem cells and/or the BMPRA-Ri population of cells are in the form of embryoid bodies. In an embodiment, the embryoid bodies were prepared by orbital shaking for about 18 hours prior to contacting the pluripotent stems with the mesoderm specifying culture composition. In an embodiment, the BMPRA and/or the BMPR1/R2 agonist is BMP4. In an embodiment, the FGF receptor agonist is or comprises FGF2. In an embodiment, the activin receptor agonist is activin A. In an embodiment, the pluripotent stem cells are embryonic stem cells or induced pluripotent stem cells, optionally human induced pluripotent stem cells.

In an embodiment, the method further comprises contacting the KDR+CD235a/b−/+ mesoderm cells with a HEC culture composition comprising VEGF and optionally FGF2 and optionally one or more hematopoietic cytokines to obtain CD34+ KDR+ hemogenic endothelial cells (HECs).

In an embodiment, method further comprises culturing the CD34+KDR+ hemogenic endothelial cells (HECs) in a primitive progenitor culture composition to obtain CD43+ hematopoietic progenitor cells.

In an embodiment, method further comprises culturing the CD43+ hematopoietic progenitor cells in the primitive progenitor culture composition to obtain primitive program lineage cells.

In an embodiment, one or more of the primitive program lineage cells are isolated.

In an embodiment, the method further comprises culturing the CD43+ hematopoietic progenitor cells with a macrophage permissive cocktail and isolating macrophage cells. In an embodiment, the method further comprises culturing the CD43+ hematopoietic progenitor cells with a mast cell permissive cocktail and isolating mast cells.

In an embodiment, the method further comprises culturing the CD43+ hematopoietic progenitor cells with an erythroid cell permissive cocktail and isolating primitive erythrocytes.

In an embodiment, wherein the ROCK inhibitor is Y-27632.

In an embodiment, wherein the BMPRA is BMP4.

In an embodiment, the method further comprises culturing the CD34+KDR+ HECs for a period of time and isolating CD34+CD43− HECs,

In an embodiment, the period of time is about 1 day.

In an embodiment, the method further comprises contacting the CD34+CD43− HECs with a multipotent progenitor (MPP) culture composition comprising a Notch agonist to obtain CD34+CD45+, optionally CD34+CD45+CD90+CD7− and/or CD34+CD45+CD90−CD7+ hematopoietic progenitor cells.

In an embodiment, the method further comprising expanding the CD34+CD45+ hematopoietic progenitor cells.

In an embodiment, the method further comprising contacting the CD34+CD45+ hematopoietic progenitor cells or the expanded CD34+CD45+ hematopoietic progenitor cells to obtain multipotent lineage cells.

In an embodiment, the one or more of the multipotent program lineage cells are isolated.

In an embodiment, macrophage cells are isolated.

In an embodiment, wherein mast cells are isolated.

In an embodiment, multiopotent program lineage erythrocytes are isolated.

In an embodiment, granulocytes are isolated

In an embodiment, T lymphocytes are isolated, for example the T lymphocytes may be gamma/delta, alpha beta, specifically Vdelta2, or a combination thereof

In an embodiment, the Notch agonist is a Notch ligand. In an embodiment, the Notch ligand is provided via a scaffold such as a culture plate or bead.

For example DL4-conjugated tissue culture plates and beads can be produced for example using methods described in Trotman-Grant, et al 2021. and can provide a serum/stroma-free method for inducing Notch signaling.

In an embodiment, one or more types of the isolated cells are resuspended in a composition.

In an embodiment, composition comprises a gel or is a sterile osmotically balanced fluid solution.

In an embodiment, the composition comprises one or more other types of cells

Also provided in an aspect is a population of cells comprising one or more types of the cells, optionally one or more types the isolated cells, generated using a method described herein.

Also provided in an aspect is a composition comprising one or more types of the cells, optionally one or more types of the isolated cells generated using a method described herein and a carrier.

In an embodiment, the composition comprises a gel, cardiomyocytes or hepatocytes.

In an embodiment, the composition comprise an osmotically balanced fluid solution.

In an embodiment, the composition is sterile.

Also provided in another aspect is a cell implant comprising a gel and one or more types of the cells optionally isolated cells generated using a method described or a composition comprising said said.

Also provided in another aspect is a mesoderm specifying culture additive comprising: a BMPR1/R2 agonist, an FGF receptor agonist and an activin receptor agonist.

In an embodiment, the specifying culture additive comprises an amount of: the BMP4 is sufficient to provide within 0.5 to about 100 ng/mL, the FGF2 is sufficient to provide within 0.5-100 ng/mL and the Activin A is sufficient to provide within 0.5 and 100 ng/ml, in a solution of about 500 mL, preferably wherein the ratio is about 10:5:6 or about 10:5:2 or within about 10:5:6 to about 10:5:2.

Also provided is a mesoderm specifying culture composition comprising: a suitable base media, a BMPR1/R2 agonist, an FGF receptor agonist, and an activin receptor agonist, optionally in concentrations or ratios described herein.

In an embodiment, the mesoderm specifying culture additive or composition is for use in a method described herein.

Also provided in another aspect is a kit comprising an additive or composition described herein.

Also provided in another aspect method of providing a subject with progenitor cells or mature cells, the method comprising administering the population of cells generated using a method described herein, a composition cell implant comprising said population of cells.

An aspect includes a method of producing a KDR+CD235a/b+ mesoderm cells capable of giving rise to T lymphoid lineage cells or cells differentiated therefrom, the method comprising:
contacting pluripotent stem cells (PSCs) with a PSC culture composition comprising a BMP receptor agonist (BMPRA) and optionally a ROCK inhibitor (Ri) to produce a BMPRA-Ri population of cells;
contacting the BMPRA-Ri population of cells with a mesoderm specifying culture composition comprising a BMPR1/R2 agonist, an FGF receptor agonist and an activin receptor agonist to produce KDR+CD235a/b+ mesoderm cells.

In an embodiment, the PSCs are contacted with the mesoderm specifying culture composition for about 3 days, at least 3 days or up to 3 days;
wherein the pluripotent stem cells and/or the BMPRA-Ri population of cells are in the form of embryoid bodies; and/or
wherein the pluripotent stem cells are embryonic stem cells or induced pluripotent stem cells, optionally human induced pluripotent stem cells.

In an embodiment, the BMPRA and/or the BMPR1/R2 agonist is BMP4; wherein the FGF receptor agonist is or comprises FGF2 and/or wherein the activin receptor agonist is activin A.

In an embodiment, the method further comprises contacting the KDR+CD235a/b+ mesoderm cells with a HEC culture composition comprising VEGF and optionally FGF2 and optionally one or more hematopoietic cytokines to obtain CD34+KDR+ hemogenic endothelial cells (HECs); and optionally further comprising:
culturing the CD34+KDR+ hemogenic endothelial cells (HECs) in a primitive progenitor culture composition to obtain CD43+ hematopoietic progenitor cells; or
culturing the CD34+ KDR+ HECs for a period of time and isolating CD34+CD43− HECs, optionally for about 1 day.

In an embodiment, the method further comprises culturing the CD43+ hematopoietic progenitor cells in the primitive progenitor culture composition to obtain primitive program lineage cells, optionally wherein the one or more of the primitive program lineage cells are isolated;
the method further comprises culturing the CD43+ hematopoietic progenitor cells with a macrophage permissive cocktail and isolating macrophage cells;
the method further comprises culturing the CD43+ hematopoietic progenitor cells with a mast cell permissive cocktail and isolating mast cells; or
the method further comprises culturing the CD43+ hematopoietic progenitor cells with an erythroid cell permissive cocktail and isolating primitive erythrocytes.

In an embodiment, the ROCK inhibitor is Y-27632, and/or wherein the BMPRA is BMP4.

In an embodiment, the method further comprises contacting the CD34+CD43− HECs with a multipotent progenitor culture composition comprising a Notch agonist to obtain CD34+CD45+, optionally CD34+CD45+CD90+CD7− and/or CD34+CD45+CD90−CD7+ hematopoietic progenitor cells and optionally expanding the CD34+CD45+ hematopoietic progenitor cells.

In an embodiment, the method further comprises contacting the CD34+CD45+ hematopoietic progenitor cells or the expanded CD34+CD45+ hematopoietic progenitor cells to obtain multipotent lineage cells and optionally isolating one or more of the multipotent program lineage cells, optionally wherein macrophage cells are isolated, mast cells are isolated, erythrocyte cells are isolated, granulocytes are isolate or T lymphocytes are isolated, optionally wherein the isolated T lymphocytes are gamma/delta, alpha/beta, T lymphocytes, optionally Vgamma2 [should this be Vdelta2 according to Michael?] T lymphocytes.

In an embodiment, the Notch agonist is a Notch ligand, optionally provided via a Notch ligand-conjugated tissue culture plate or bead.

In an embodiment, one or more types of the isolated cells are resuspended in a composition, optionally wherein the composition comprises a gel or is a sterile osmotically balanced fluid solution and/or wherein the composition comprises one or more other types of cells.

A further aspect includes a population of cells or composition comprising one or more types of the cells, optionally one or more types the isolated cells, described herein wherein the composition comprises a carrier, a gel, and/or an osmotically balanced fluid solution, optionally where the composition is sterile, optionally wherein the population of cells or composition comprises cardiomyocytes or hepatocytes.

Another aspect includes cell implant comprising a gel or a scaffold, optionally a pouch, and one or more types of isolated cells prepared according to the method described herein, or the population of cells or composition described herein.

A further aspect includes a mesoderm specifying culture additive or culture composition or kit comprising:
a BMPR1/R2 agonist, optionally BMP4,
an FGF receptor agonist, optionally FGF2, and
an activin receptor agonist, optionally Activin A;
   optionally, wherein the amount of: BMPR1/R2 agonist, optionally BMP4 is sufficient to provide within 0.5 ng/mL to about 100 ng/mL, the FGF receptor agonist, optionally FGF2 is sufficient to provide within 0.5 ng/mL-100 ng/mL and the activin receptor agonist, optionally Activin A is sufficient to provide within 0.5 ng/mL-100 ng/ml, in a solution of about 500 mL, preferably wherein the ratio of BMP4 to FGF2 to Activin A is about 10:5:6 or about 10:5:2 or within about 10:5:6 to about 10:5:2, or
   wherein the mesoderm specifying culture composition further comprises a hematopoetic progenitor suitable base media.

In an embodiment, the mesoderm specifying culture additive or composition is for use in a method described herein.

Also provided is use of the population of cells or compositions, cell implants, mesoderm specifying culture additives or compositions described herein for preparing a medicament.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### DRAWINGS

The drawings included herewith are for illustrating various examples of articles and methods of the present specification and are not intended to limit the scope of what is taught in any way. In the drawings:

FIG. 1 Induction of KDR⁺CD235a/b⁺ mesoderm. A) Schematic of mesoderm specification from hPSCs through the addition of BMP4, FGF2 and Activin A (A) on day 1 of differentiation. B) Representative flow cytometric analysis of KDR and CD235a/b expression on days 3 and 4 of differentiation. C) Quantification of the proportion of KDR⁺CD235a/b⁺ cells on day 3 of differentiation (n=3). ANOVA. *P<0.05 and **P<0.01 versus cultures induced with 6 ng/mL of Act A. D) Quantification of the proportion of KDR⁺CD235a/b⁺ cells on day 4 of differentiation (n=3). ANOVA. *P<0.05, ***P<0.001 and ****P<0.0001 versus cultures induced with 6 ng/mL of Act A.

FIG. 2 Emergence of the primitive program from hPSCs. A) Schematic of hematopoietic differentiation from hPSCs through the addition of BMP4, FGF2 and ACT A followed by culture in the presence of VEGF, FGF2 and hematopoietic cytokines. B) Representative flow cytometric analysis of CD43 and CD45 expression between days 6 and 15 of differentiation. C) Quantification of the number of total, CD43⁺ and CD45⁺ cells generated from 500,000 input H1 hESCs in the cultures at the indicated days (n=4). ANOVA. *P<0.05, **P<0.01 and ****P<0.0001 versus the indicated population on day 6 of differentiation. D) Colony-forming progenitor numbers generated between days 6 and 15 of differentiation based on an input of 500,000 H1 hESCs (n=3). ANOVA. **P<0.01, ***P<0.001 and ****P<0.0001 versus the indicated lineage on day 9 of differentiation (black=all colonies). E) Distribution of erythroid progenitors generated between days 6 and 15 of differentiation (n=3). ANOVA. **P<0.01, ***P<0.001 and ****P<0.0001, and ##P<0.01 and ####P<0.0001 versus small and large erythroid colony morphologies, respectively. F) RT-qPCR analysis of the percentage of HBE, HBG and HBB β globin expression of small erythroid colonies (n=3). ANOVA. ****P<0.001 versus the indicated β globin gene.

FIG. 3 Hematopoietic potential of KDR⁺ mesoderm. A) Gating strategy used to isolate KDR⁺ mesoderm based on CD235a/b expression on day 4 of differentiation. B) Representative flow cytometric analysis of CD43 expression over 5 days in populations generated from the KDR⁺ fractions. C) Quantification of the number of total and CD43+ cells generated from 62,500 day 4 KDR⁺ cells over 5 days of culture (n=3). ANOVA. **P<0.01 versus the indicated sample. D) Colony-forming progenitor number after 5 days of culture of 62,500 day 4 KDR⁺ cells (n=3). ANOVA. not significant. E) BL-CFC frequency of the KDR⁺ populations on day 4 of differentiation (n=3). t-test. not significant.

FIG. 4 Characterization of KDR⁺CD34+ cells at day 5 of differentiation. A) Representative flow cytometric analysis of CD43 expression between days 4 and 6 of differentiation. B) Representative flow cytometric analysis of KDR and CD34 expression between days 4 and 6 of differentiation. C) Representative flow cytometric analysis of KIT, CD144, CD31 and CD45 expression in the KDR⁺CD34⁺ population (gray) at day 5 of differentiation. Black line: unstained cells. D) RT-qPCR analysis of SCL/TAL1 and RUNX1a/b expression in the KDR⁺CD34⁺ and unsorted populations on day 5 of differentiation (n=3). t-test. *P<0.05.

FIG. 5 The primitive program transitions through a hemogenic endothelial cell intermediate. A) Gating strategy (grey) used for the isolation the KDR⁺CD34⁺ population on day 5 of differentiation. B) Representative flow cytometric analysis of CD34 and CD43 expression in populations generated from the day 5 KDR⁺CD34⁺ isolated cells. C) Quantification of the number of total and CD43⁺ cells generated from 62,500 day 5 KDR⁺CD34⁺ cells over 7 days of culture (n=4-5). ANOVA. *P<0.05, **P<0.01 and ****P<0.0001 versus the indicated population after 1 day of culture. D) Colony-forming progenitor numbers generated from 62,500 day 5 KDR⁺CD34⁺ cells over 7 days of culture (n=4-5). ANOVA. *P<0.05 and **P<0.01 versus the indicated lineage after 4 days of culture (black=all colonies). E) Representative phase-contrast images of monolayer cultures derived from the day 5 KDR⁺CD34⁺ population over 4 days of culture. Scale bar=50 μm. F) Quantification of the number of total and CD43⁺ cells over 4 days in monolayer cultures generated from 20,000 day 5 KDR⁺CD34⁺ cells (n=3). t-test. *P<0.05 versus the indicated population after 2 days of culture.

FIG. 6 NOTCH signaling during the development of the primitive program. A) RT-qPCR analysis of NOTCH receptor expression in the day 5 KDR⁺CD34⁺ and pre-sorted populations following 5 days of culture (n=3). t-test. *P<0.05, **P<0.01 and ***P<0.001. B) RT-qPCR analysis of NOTCH target gene expression over 7 days of monolayer culture of the day 5 KDR⁺CD34⁺ cells (n=3). ANOVA. *P<0.05, **P<0.01 and ***P<0.001 versus day 1 of culture.

FIG. 7 NOTCH signaling is required for the generation of the hematopoietic cells of the primitive program. A) RT-qPCR analysis of NOTCH target gene expression over 4 days of monolayer culture of the day 5 KDR⁺CD34⁺ cells in the presence or absence of the NOTCH inhibitor, GSI (n=3). ANOVA. **P<0.01 versus the stage-matched sample. B) Representative phase-contrast images of populations generated from the day 5 KDR⁺CD34⁺ cells cultured in the presence or absence of the NOTCH inhibitor, GSI. C) Representative flow cytometric analysis of CD34 and CD43 expression of populations generated from day 5 KDR⁺CD34⁺ cells cultured in monolayer in the presence or absence of the NOTCH inhibitor, GSI. D) Quantification of the number of total and CD43⁺ cells generated from 20,000 day 5 KDR⁺CD34⁺ cells cultured in the presence or absence of the NOTCH inhibitor, GSI (n=3). ANOVA. ****P<0.0001 versus the stage-matched population. E) Number of colony-forming progenitors generated from 20,000 day 5 KDR⁺CD34⁺ cells cultured for the indicated time in the presence or absence of the NOTCH inhibitor, GSI (n=3). ANOVA. ****P<0.0001.

FIG. 8 Characterization of CD34⁺CD43⁻ cells at day 6 of differentiation. A) Representative flow cytometric analysis of CD34 and CD43 expression on day 6 of differentiation. B) Representative flow cytometric analysis of KDR, KIT, CD144, CD31 and CD45 expression on the day 6 CD34⁺CD43⁻ population (gray). Black line: unstained cells. C) RT-qPCR analysis of SCL/TAL1 and RUNX1a/b expression in the day 6 CD34⁺CD43⁻ and pre-sort populations (n=6). t-test. ***P<0.001.

FIG. 9 Separation of two hematopoietic programs at day 6 of differentiation based on CD34 and CD43 expression. A) Gating strategy for the FACS based isolation of the day 6 CD43⁺ and CD34⁺CD43⁻ populations. B) Representative flow cytometric analysis of CD34 and CD43 expression on the populations generated from CD43⁺ and CD34⁺CD43⁻ cells isolated on day 6 of differentiation. C) Quantification of the number of total and CD43⁺ cells generated from 62,500 day 6 CD43⁺ or CD34⁺CD43⁻ isolated cells (n=5). t-test and ANOVA. **P<0.01, ***P<0.001 and ****P<0.0001 versus the stage-matched sample or versus after 1 day of culture within the same sample, as indicated. D) Colony-forming progenitor numbers in populations generated from 62,500 day 6 CD43⁺ or CD34⁺CD43⁻ cells (n=5). t-test and ANOVA. *P<0.05, **P<0.01 and ****P<0.0001 versus the stage-matched sample or versus after 1 day of culture within the same sample, as indicated (black=all colonies). E) Distribution of lineages observed in D. ANOVA. **P<0.01, ***P<0.001 and ****P<0.0001 versus the stage-matched sample. F) Distribution of erythroid progenitors observed in D. ANOVA. *P<0.05, **P<0.01 and ***P<0.001, and #P<0.05 and ##P<0.01 versus small and large erythroid colony morphologies, respectively.

FIG. 10 CD34⁺CD43⁻ HECs give rise to CD45+ hematopoietic progenitors. A) Representative flow cytometric analysis of CD34 and CD45 expression on populations generated from the day 6 CD43⁺ and CD34⁺CD43⁻ cells. B) Quantification of the proportion of CD45⁺ cells over 6 days in the populations generated from the day 6 CD43⁺ and CD34⁺CD43⁻ cells (n=5). ANOVA. ***P<0.001 versus the stage-matched population. C) Quantification of the number of CD45⁺ cells over in the populations generated from the day 6 CD43⁺ and CD34⁺CD43⁻ cells (n=5). ANOVA. **P<0.01 versus the stage-matched population. D) Gating strategy used for FACS-based isolation of the CD34 and CD45 populations generated from the day 6 CD34⁺CD43⁻ cells. Cells were cultured as aggregates for 5 days. E) Colony-forming progenitor frequency in the isolated CD34 and CD45 populations shown in D (n=3). ANOVA. *P<0.05 and ****P<0.0001 versus the indicated lineage and population. F) Distribution of myeloid progenitors observed in E. G) RT-qPCR analysis of the percentage of HBE, HBG and HBB R globin expression of erythroid colonies derived from the day 6 CD43⁺ population after 6 days of culture and CD34⁺CD45⁺KIT⁺ cells generated after 5 days of culture of the day 6 CD34⁺CD43⁻ population (n=4-12). **P<0.01 and **'P<0.0001 versus the indicated β globin or sample.

FIG. 11 CD34⁺CD43⁻ HECs have T lymphoid potential. A) Representative flow cytometric analysis of CD45, CD56, CD5, CD7, CD4 and CD8 expression on cells derived from the day 6 CD43⁺ or CD34⁺CD43⁻ populations following 30 days of culture with OP9-DL4 cells. B) Representative flow cytometric analysis of CD3, TCRαβ and TCRγδ expression on the CD45⁺ cells generated from the day 6 CD34⁺CD43⁻ population cultured with OP9-DL4 cells for 40 days. C) Quantification of the proportion of CD3⁺TCRαβ⁺ and CD3⁺ TCRγδ⁺ cells in the day 40 CD45⁺ populations described in B (n=4). t-test. not significant.

FIG. 12 KDR⁺CD235a/b⁺ mesoderm gives rise to the T lymphoid lineage. A) Gating strategy used for the FACS-based isolation of the KDR⁺CD235a/b⁺ population on day 4 of differentiation. B) Representative flow cytometric analysis of CD34 and CD43 expression on the day 4 KDR⁺CD235a/b⁺-derived population following 3 days of culture. C) Representative flow cytometric analysis of CD4 and CD8 expression on the CD45⁺CD56⁻ population generated from the CD34⁺CD43⁻ cells (shown in B) following culture for 30 days with OP9-DL4 cells.

FIG. 13 Analyses of T lymphocytes. A) Representative flow cytometric analysis of CD3 expression in CD45⁺ populations generated from the culture of hPSC-derived day 6 CD34⁺CD43⁻ cells or CD34⁺ cord blood cells with OP9-DL4 cells for the indicated number of days. B) Quantification of the proportion of CD3⁺ cells in CD45⁺ populations generated from the culture of hPSC-derived day 6 CD34⁺CD43⁻ cells or CD34⁺ cord blood cells with OP9-DL4 cells for the indicated number of days (n=3). ANOVA. ***P<0.001 and ****P<0.0001 versus day 25 of culture. #P<0.05 and ##P<0.01 versus the indicated stage-matched sample. C) Representative flow cytometric analysis of TCRγδ and TCRαβ expression in CD45⁺CD3⁺ populations generated from the culture of hPSC-derived day 6 CD34⁺CD43⁻ cells or CD34⁺ cord blood cells with OP9-DL4 cells for the indicated number of days. D) Quantification of the proportion of TCRγδ ⁺ and TCRαβ⁺ cells in CD45⁺CD3⁺ populations generated from the culture of hPSC-derived day 6 CD34⁺CD43⁻ cells or CD34⁺ cord blood cells with OP9-DL4 cells for the indicated number of days (n=3). ANOVA. *P<0.05, ***P<0.001 and ****P<0.0001 versus day 25 of culture. #P<0.05, ##P<0.01 and ####P<0.0001 versus the indicated stage-matched sample. E) Representative flow cytometric analysis of TCRVδ2 and TCRVγ9 expression in CD45⁺CD3⁺ populations generated from the culture of hPSC-derived day 6 CD34⁺CD43⁻ cells or CD34⁺ cord blood cells with OP9-DL4 cells for the indicated number of days. F) Representative flow cytometric analysis of TCRVδ1 expression in CD45⁺CD3⁺ populations generated from the culture of hPSC-derived day 6 CD34⁺CD43⁻ cells or CD34⁺ cord blood cells with OP9-DL4 cells for the indicated number of days. G) Quantification of the proportion of TCRVδ2⁺ and TCRVδ1⁺ cells in CD45⁺CD3⁺ populations generated from the culture of hPSC-derived day 6 CD34⁺CD43⁻ cells or CD34⁺ cord blood cells with OP9-DL4 cells for the indicated number of days (n=3). ANOVA. **P<0.01, and ****P<0.0001 versus the indicated lineage-matched sample. #P<0.05, ###P<0.01 and ####P<0.0001 versus the indicated stage-matched sample.

FIG. 14 Analyses of T lymphoid progenitors. A) Representative flow cytometric analysis of CD34, CD7 and CD7 expression on the CD45⁺ populations generated from hPSC-derived day 6 CD34⁺CD43⁻ cells or CD34⁺ cord blood following 12 days of culture with OP9-DL4 cells. B) Quantification of the proportion of CD5⁺ cells in either the CD34⁺CD7⁺ and CD34⁻CD7⁺CD45⁺ populations generated from hPSC-derived day 6 CD34⁺CD43⁻ cells or CD34⁺ cord blood following 12 days of culture with OP9-DL4 cells (n=4). ANOVA. *P<0.05 and ****P<0.0001 versus the indicated population.

FIG. 15 Characterization of the day 6 CD43⁺ and CD34⁺CD43⁻ HEC-derived hematopoietic cells. A) Representative flow cytometric analysis of CD34, CD45, CD90 and CD7 expression on the day 6 CD34+CD43⁻-derived population generated following 5 days of culture with OP9-DL4 cells. B) Representative flow cytometric analysis of CD45, CD90 and CD7 expression on the day 6 CD43⁺ population. Black line: unstained cells.

FIG. 16 The expression of CD90 and absence of CD7 marks a CD34⁺CD45+ hematopoietic progenitor population with multilineage potential. A) Limiting dilution analysis of NK cell and T lymphoid progenitor frequency of the CD34⁺CD45⁺CD90⁺CD7⁻ and CD34⁺CD45⁺CD90⁻CD7⁺ populations (n=2). B) Quantification of the number of CD45⁺ cells generated from 25 CD34⁺CD45⁺CD90+CD7⁻ or CD34⁺CD45⁺CD90⁻CD7⁺ cells following 4 days of culture with HUVEC-E4ORF1 cells. The numbers within the graph indicate the average fold change in cell number (n=9). t-test. **P<0.01. C) Quantification of the number of CD34⁺CD45⁺ cells generated from 25 CD34⁺CD45⁺CD90⁺CD7⁻ or CD34⁺CD45⁺CD90⁻CD7⁺ cells following 4 days of culture with HUVEC-E4ORF1 cells. The numbers within the graph indicate the average fold change in cell number (n=9). t-test. **P<0.01. D) Quantification of the number of CD45⁺ cells generated from 25 CD34⁺CD45⁺CD90⁺CD7⁻ or CD34⁺CD45⁺CD90⁻CD7⁺ cells following 8 days of culture with OP9-DL4 cells. The numbers within the graph indicate the average fold change in cell number (n=12). t-test. not significant. E) Heatmap representing wells that gave rise to NK cell, T lymphoid, myeloid and erythroid progeny (grey). F) Quantification of the proportion of wells that generated all 4 lineages shown in E.

FIG. 17 The CD34⁺CD45⁺CD90+CD7⁻ population contains multipotent hematopoietic progenitors. A) Quantification of the proportion of wells seeded with single CD34⁺CD45⁺CD90⁺CD7⁻ cells that generated a hematopoietic clone. B) Representative flow cytometric analysis of CD56, CD7, CD4 and CD8 expression and brightfield image identifying the NK cell, T lymphoid, myeloid and erythroid lineages generated from a single CD34⁺CD45⁺CD90⁺CD7⁻ cell. C) Summary the NK cell, T lymphoid, myeloid and erythroid lineage potential of all cells that gave rise to a hematopoietic clone (n=60). D) RT-qPCR analysis of the percentage of HBE, HBG and HBB β globin expression in erythroid colonies generated from the hPSC-derived primitive, MPP, definitive and cord blood progenitors (n=5-35). ANOVA. **P<0.01 and ****P<0.0001 relative to the indicated sample.

FIG. 18 Engraftment potential of hPSC-derived multipotent hematopoietic progenitors. A) Representative flow cytometric analysis of human CD45, CD19 and CD11 b expression on cells in the bone marrow of recipients 4 weeks following transplantation of CD34⁺ cord blood cells. B) Representative flow cytometric analysis of human CD45 expression on cells in the bone marrow of recipients 4 weeks following transplantation of hPSC-derived CD34⁺CD45⁺CD90+CD7⁻ multipotent progenitor cells. C) Quantification of the proportion of human CD45⁺ cells in the mouse bone marrow 4 weeks after transplantation (n=2-4).

FIG. 19 The primitive and multipotent progenitor programs do not develop from ALDH⁺ progenitors. A) Representative flow cytometric analysis of ALDH activity in the CD34⁺CD45⁺CD90⁺CD7⁻ population generated from day 6 CD34⁺CD43⁻ cells following 5 days of culture with OP9-DL4 cells. B) Representative flow cytometric analysis of CD235a/b expression and ALDH activity in the primitive/MPP and definitive populations on days 3 and 4 of differentiation. C) Representative flow cytometric analysis of CD34 expression and ALDH activity in the prim itive/MPP and definitive populations on days 5 and 6 of differentiation. D) RT-qPCR analysis of ALDH1A2 and CYP26A1 expression between days 0 and 6 of differentiation in primitive/MPP-induced populations (n=5). ANOVA. ****P<0.0001 versus the indicated sample.

FIG. 20 Generation of the yolk sac hematopoietic lineage from CHOP10WT iPSCs. A) Representative flow cytometric analysis of KDR and CD235a/b expression on day 4 of differentiation. B) Quantification of the proportion of KDR⁺CD235a/b+ cells on day 4 of differentiation (n=3). ANOVA. *P<0.05, **P<0.01, ***P<0.001 and ****P<0.0001 versus the indicated sample. C) Representative flow cytometric analysis of CD43 and CD45 expression on populations between days 6 and 12 of differentiation. D) Quantification of the number of total and CD43⁺and CD45⁺ all cells in the populations between days 6 and 12 of differentiation (n=3). ANOVA. *P<0.05, **P<0.01 and ****P<0.0001 versus the indicated population on day 6 of differentiation.

FIG. 21 Hematopoietic potential of KDR⁺CD235a/b⁺ mesoderm generated from CHOP10WT iPSCs. A) Gating strategy used for FACS-based isolation of the KDR⁺CD235a/b⁺ population on day 4 of differentiation. B) Representative flow cytometric analysis of CD34 and CD43 expression on aggregate populations generated from the day 4 KDR⁺CD235a/b⁺ cells. C) Quantification of the number of total and CD43+ cells generated from 62,500 day 4 KDR⁺CD235a/b⁺ cells (n=3). ANOVA. not significant. D) Colony-forming progenitor number generated from 62,500 day 4 KDR⁺CD235a/b⁺ cells following 5 days of culture (n=3). ANOVA. not significant. E) Representative flow cytometric analysis of CD4 and CD8 expression on the CD45⁺CD56⁻ population generated from day 4 KDR⁺CD235a/b⁺ cells following 30 days of culture with OP9-DL4 cells.

FIG. 22 is a schematic showing a model of human yolk sac hematopoietic development using pluripotent stem cells.

FIG. 23 Macrophage differentiation from yolk sac hematopoietic cell-like progenitors. A) Schematic of macrophage differentiation from hematopoietic progenitor cells through the addition of 1L3, SCF and MCSF followed by culture in the presence of MCSF for two weeks. B) Representative flow cytometric analysis of CD45, CD68, CD64, CD163, CD14 and CD11b expression in populations generated after two weeks of culture in the conditions described in A.

### DESCRIPTION OF VARIOUS EMBODIMENTS

Various processes, products and uses will be described below to provide an example of an embodiment of the claimed subject matter. No embodiment described below limits any claim and any claim may cover processes or products that differ from those described below. The claims are not limited to products or processes having all of the features of any one products or processes described below or to features common to multiple or all of the methods or products described below. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

The progenitors of the yolk sac-derived hematopoietic progenitors cells can not be isolated after birth. For yolk sac-like hematopoietic cells to be tested and realized, PSCs represent the only source to prepare these progenitors.

The inventors had previously shown that appropriately staged inhibition of Wnt signaling induces a population of KDR+CD235a+ mesoderm from hPSCs that displays erythroid and myeloid potential indicative of yolk sac hematopoiesis (Sturgeon et al., 2014). This mesoderm, however, did not give rise to the T lymphoid lineage, suggesting that it does not recapitulate the full developmental potential of the mouse yolk sac.

To overcome this hurdle and access these progenitor populations, the inventors have herein established a developmental biology-guided protocol to generate the yolk sac hematopoietic programs from human pluripotent stem cells (hPSCs) in vitro. The inventors have determined that the human primitive program transitions through a progenitor, known as the hemogenic endothelial cell (HEC), prior to the generation of the first hematopoietic cells. Coincident with the emergence of primitive hematopoietic cells, the inventors demonstrate that a second population of HECs gives rise to the progenitors of the EMP program. The inventors show that this second HEC population also harbours T lymphoid potential indicative of the development of the LMP program. Clonal analyses revealed that the EMP and LMP programs derive from a common hematopoietic progenitor, defining in the human, a unified yolk sac program that develops from a multipotent cell. The in vitro methods provide a progenitor source for disease modeling and regenerative medicine applications, including the therapeutic use of tissue-resident immune cells.

In particular the inventors show that the combination of signaling activities for example Activin A, BMP4 and FGF2 signaling induces KDR+CD235a/b+ mesoderm that gives rise to the primitive and EMP hematopoietic programs. Both programs were found to transition through a hemogenic endothelial cell (HEC) intermediate providing evidence that all hematopoietic programs share this transition in common. Detailed analyses showed that the HECs that give rise to EMP hematopoiesis also generate γδ and αβ T cells, including the Vδ2+ lineage. Through clonal analyses, it is demonstrated herein that this HEC-derived population contains CD34+CD45+CD90+CD7− multipotent hematopoietic progenitor capable of generating erythroid, myeloid, NK and T cell progeny. Analyses of the mouse yolk sac EMP population revealed that it also has T cell potential and contains bipotent and multipotent progenitors that can generate myeloid, erythroid and T cell progeny.

### I. DEFINITIONS

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells, “an agonist” includes a single agonist or a combination of agonists etc. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

Terms of degree such as “about”, “substantially”, and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “isolated” as used herein with respect to cells, means substantially free of culture media and/or enriching for a particular cell type based on for example cell surface receptors, for example by FACS or MACS as described herein. For example isolating can include a population that is about 80%, 85%, 90%, 95%, 96%, 97%, 98% or 99% pure.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “pluripotent stem cell” as used herein refers to a cell with the capacity to differentiate into cell of the three germ cell layers. Pluripotency is also evidenced by the expression of embryonic stem (ES) cell markers. Suitable pluripotent cells for use herein include human embryonic stem cells (hESCs) and human induced pluripotent stem (iPS) cells.

The term “hematopoietic progenitor cells” as used herein refers to any cell that expresses CD43 and/or CD45. For example, primitive hematopoietic progenitor cells are defined by the expression of CD43 whereas multipotent hematopoietic progenitors or MPPs are defined by the expression of CD45. These cells are highly enriched in the population marked as CD34+CD45+, including CD34+CD45+CD90−CD7+ and/or CD34+CD45+CD90+CD7−.

The term “T lymphoid progenitors” as used herein refers to cells that are enriched in CD45+ populations defined by the expression of two of the following CD34, CD7 and CD5 or the co-expression of CD4 and CD8.

Blood cells that can be produced by the present methods can be defined and/or isolated by by cell surface markers. For example, mature T lymphocytes are defined by the expression of CD45, CD3 and TCR; NK cells are defined by the expression of CD45 and CD56; macrophages are defined by the expression of CD45, CD68, CD64, CD163, CD11b and/or CD14; erythroid progenitors are defined by expression of CD43 and CD235a/b, mast cells and mast cell progenitors are defined by the expression of CD45 and KIT; and granulocytes are defined by the expression of CD45, CD15 and CD31.

The term “hematopoietic cytokines” as used herein refers cytokines and growth factors that promote differentiation and includes but is not limited to of cytokines: IL-6, IL-7 IL-11, SCF, EPO, IGF1, SCF, FLT3L, as well as GM-CSF, M-CSF and the like. Other cytokines or growth factors and other combinations than described herein can also be used.

The term “BMPRA” or “BMPR1/R2 agonist” as used herein refers to any molecule that can activate BMP signaling through the receptor and induce SMAD phosphorylation. This includes, but is not limited to BMP4, BMP2 and BMP7 as well as active conjugates and/or active fragments thereof, preferably human BMP4 an active conjugates and active fragments thereof.

The term “BMP4” refers to Bone Morphogenetic Protein 4, and includes but is not limited to human BMP4 (e.g. Uniprot accession number P12644), as well as non-human cytokines such as chimp BMP4, and all naturally occurring variants thereof and includes active conjugates and/or active fragments of any of thereof that can activate BMP signaling.

The term “FGF receptor agonist” as used herein refers to any molecule that can activate FGF signaling through an FGF receptor. This includes, but is not limited to FGF2.

The term “FGF2” refers to Fibroblast Growth Factor 2 also referred to as basic FGF (bFGF), and includes but is not limited to human FGF2 (e.g. Uniprot accession number P09038), as well as non-human cytokines, such as chimp FGF2, and all naturally occurring variants thereof and includes active conjugates and/or active fragments of any of thereof that can activate FGF signaling.

The term “activin receptor agonist” as used herein refers to any molecule that can activate Nodal signaling through one of its receptors, ALK4 (Uniprot accession number: P36896), ALK7 (Uniprot accession number: Q8NER5), ACTRIIA (Uniprot accession number: P27037) and/or ACTRIIB (Uniprot accession number: Q13705). This includes, but is not limited to Activin A and/or Nodal, preferably human Activin A and/or human Nodal as well as active conjugates and/or active fragments of any of thereof.

The term “Activin A” or “Act A”, refers to for example all forms of Activin A, including human Activin A (Uniprot accession number: P08476) as well as well as non-human cytokines, and all naturally occurring variants thereof and includes active conjugates and/or active fragments of any of thereof that can activate Nodal signaling.

The term “Notch agonist” as used herein includes any molecule that can activate Notch signaling. This includes, but is not limited to Notch ligands, Delta-like (DL) 1, 2 and 4, and Jagged (Jag) 1, 2 as well as well as non-human proteins, and all naturally occurring variants thereof and includes active conjugates and/or active fragments of any of thereof.

The term “Notch” as used herein includes for example NOTCH1 (Uniprot accession number: P46531), NOTCH2 (Uniprot accession number: Q04721), NOTCH3 (Uniprot accession number: Q9UM47) and/or NOTCH4 (Uniprot accession number: Q99466) and naturally occurring variants thereof, preferably human Notch.

The term “PSC culture composition comprising a BMP receptor agonist (BMPRA) and a optionally a ROCK inhibitor” as used herein refers to a base media suitable for pluripotent stem cells comprising a BMP receptor agonist (BMPRA) such as BMP4 and a ROCK inhibitor such as Y-27632. Other ROCK inhibitors can also be used, such as GSK429286A, Fasudil and Thiazovivin. It may include one or more other components for example, one or more other components described herein, for example in Example 1.

The term “mesoderm specifying culture composition comprising a BMP4R1/R2 agonist, an FGF receptor agonist and an activin receptor agonist” as used herein refers to a composition comprising a base media such as StemPro-34 and a BMP4R1/R2 agonist, such as BMP4, a FGF receptor agonist, such as FGF2 and an activin receptor agonist, such as Activin A. It may include one or more other components for example, one or more other components described herein, for example in Example 1.

The term “HEC culture composition comprising VEGF” as used herein refers to a composition comprising a base media such as StemPro-34 or alpha-MEM and VEGF, and optionally comprises a FGF agonist such as FGF2 and one or more hematopoietic cytokines such as IL-6 and/or IL-11. It may include one or more other components for example, one or more other components described herein, for example in Example 1.

The term “VEGF” refers to Vascular Endothelial Growth Factor family members, for example human VEGF family members including VEGFA (e.g. Uniprot accession number P15692), as well as non-human cytokines, and all naturally occurring variants thereof and includes active conjugates and/or active fragments of any of thereof that can activate VEGF signaling.

The term “IL-6” refers to Interleukin-6, for example human IL-6 (e.g. Uniprot accession number: P05231), as well as non-human cytokines, and all naturally occurring variants thereof, active conjugates and/or active fragments of any of thereof that can activate IL-6 signaling.

The term “IL-11” refers to Interleukin-11, for example human IL-11 (e.g. Uniprot accession number: P20809), as well as non-human cytokines and all naturally occurring variants thereof, and includes active conjugates and/or active fragments of any of thereof that can activate IL-11 signaling.

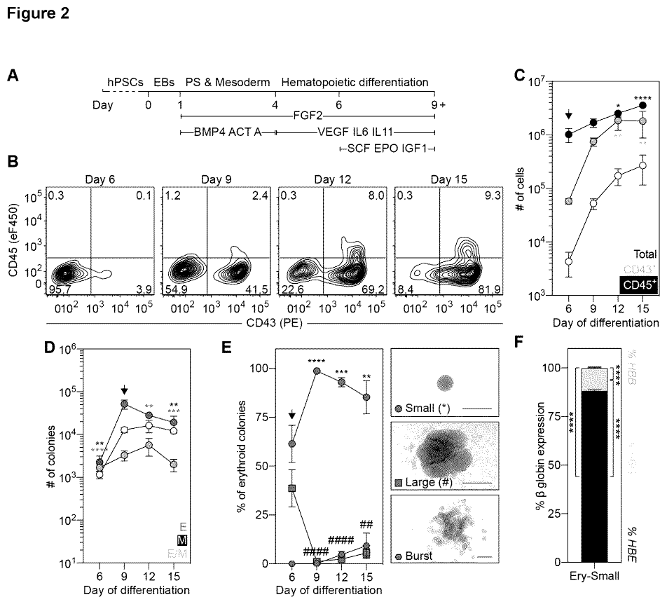

The term “day 6 cells” or “day 6 differentiation” as used herein refers to cells that comprise CD34+CD43−, for example at least 15%, at least 25%, at least 30% or between 15% and 60%. and can be differentiated to progress along the multipotent progenitor pathway or cells that are CD34+CD43+ and are primitive. As shown in FIG. 2A, day 6 cells refer to cells that are 6 days post differentiation starting from PSCs as described herein. CD34+CD43− cells for example may also be isolated on day 7 of differentiation.

The term “primitive progenitor culture composition” as used herein refers to a composition comprising a base media suitable for hematopoietic progenitor cells and includes VEGF and optionally FGF2 one or more hematopoietic cytokines. The primitive progenitor culture composition can be the same composition as the HEC culture composition.

The term “EPO” refers to Erythropoietin, including for example human EPO (e.g. Uniprot accession number: P01588), as well as non-human cytokines, and all naturally occurring variants thereof, and includes active conjugates and/or active fragments of any of thereof that can activate EPO signaling.

The term “IGF1” refers to Insulin-like Growth Factor 1, for example human IGF1 (e.g. Uniprot accession number: P05019), as well as non-human cytokines, and all naturally occurring variants thereof, and includes active conjugates and/or active fragments of any of thereof that can activate IGF1 signaling.

The term “SCF”) refers to Stem Cell Factor, also known as KIT ligand (KL), for example human SCF (e.g. Uniprot accession number: P21583), as as well as non-human cytokines, and all naturally occurring variants thereof, and includes active conjugates and/or components thereof that can activate KIT signaling.

For example, the base media can for example be commercially available StemPro34 (ThermoFisher Scientific, 10639011) used as supplied or partially diluted with IMDM (ThermoFisher Scientific, 12200036) further supplemented with ITS-X (ThermoFisher Scientific, 51500056) additional glutamine, ascorbic acid, monothioglycerol and transferrin. Other base medias such as GMEM, DMEM, RPMI, STEMdiff APEL2, STEMspan SFEM II, alpha-MEM and X-VIVO. Other supplements can also be used.

The term “a multipotent progenitor culture composition” or “MPP culture composition” as used herein refers to a composition comprising a base media such as StemPro-34, or alpha-MEM and one or more hematopoietic cytokines. For example, the multipotent progenitor culture composition can be used when using a Notch ligand providing cell source, such as OP9-DL4 cells. In such examples, the multipotent progenitor culture composition may comprise serum and hematopoietic cytokines such as SCF, IL7 and FLT3L.

The term “IL7” refers to Interleukin-7, for example human IL7 (Uniprot accession number: P16871), as as well as non-human cytokines, and all naturally occurring variants thereof and includes active conjugates and/or active fragments thereof that can activate IL7 signaling.

The term “FLT3L” refers to Fms-related Tyrosine Kinase 3 ligand, for example human FLT3L (e.g. Uniprot accession number: P49771), as as well as non-human cytokines, and all naturally occurring variants thereof and includes active conjugates and/or active fragments thereof that can activate FLT3 signaling.

The term “mast lineage cells” refers to cells that are defined by, but not limited to the expression of CD45 and KIT.

The term “NK lineage cells” refers to cells that are defined by, but not limited to the expression of CD56, for example CD45, CD7 and CD56.

The term “primitive erythrocyte lineage cells” refers to cells that are defined by, but not limited to the expression of CD43 and CD235a/b.

The term “macrophage lineage cells” refers to cells that are defined by, but not limited to the expression of CD45, and one or more of CD64, CD68, CD163, CD11 b and CD14.

The term “granulocyte lineage cells” refers to cells that are defined by, but not limited to the expression of CD45 and CD15 and optionally CD31.

As used herein, the term carrier or “pharmaceutically acceptable carrier” is intended to include any and all solvents, dispersion media, coatings, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions, dextrose solution, and 5% human serum albumin and bovine serum albumin (BSA).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% or ±10% of the modified term if this deviation would not negate the meaning of the word it modifies.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. METHODS AND COMPOSITIONS

Analyses of model organism development have demonstrated that embryonic hematopoiesis consists of distinct programs that differ in spatiotemporal organization and lineage potential. Although long thought to primarily fulfil the hematopoietic requirements, such as the oxygen demand, of the rapidly developing early embryo, studies over the past decade have provided strong evidence that the progeny of the yolk sac hematopoietic programs contribute to tissue-resident immune cell populations in the fetus and adult. These immune cell populations serve essential tissue-specific homeostatic functions and their dysregulation can lead to disease. Described herein are methods and compositions for the generation and regulation of the human primitive, EMP and LMP hematopoietic programs using hPSC differentiation. The methods described provide an efficient protocol to generate the hematopoietic progenitors of these embryonic hematopoietic programs from KDR+CD235a/b+ mesoderm. The inventors have uncovered regulatory roles for NOTCH in the generation of the human primitive program. In addition, it is demonstrated herein that KDR+CD235a/b+ mesoderm can give rise to progenitors with T lymphoid potential and show that this lineage develops from a multipotent hematopoietic progenitor with NK cell, T lymphoid, EMP erythroid and myeloid potential. Together, these findings link the human EMP and LMP programs and provide evidence for a common yolk sac-derived multipotent progenitor (MPP) program in the human

The inventors have identified in vitro culture methods for making human yolk sac like hematopoietic cells from pluripotent stem cells.

Accordingly, one aspect includes a method of producing a KDR+CD235a/b+ mesoderm cells capable of giving rise to T lymphoid lineage cells or cells differentiated therefrom, the method comprising:

contacting pluripotent stem cells (PSCs) with a with a PSC culture composition comprising a BMP receptor agonist (BMPRA) and optionally a ROCK inhibitor (Ri) to produce a BMPRA-Ri population of cells; and contacting the BMPRA-Ri population of cells with a mesoderm specifying culture composition comprising a BMPR1/R2 agonist, an FGF receptor agonist and an activin receptor agonist to produce KDR+CD235a/b+ mesoderm cells.

In various embodiments, the PSC culture composition includes the Ri.

The contacting or treatment with mesoderm specifying culture composition directs cells to proceed through the primitive streak (PS) and produce mesoderm.

The population of cells produced by the specifying culture composition include KDR+CD235a/b+ and KDR+CD235a/b−cells. As demonstrated herein, the KDR+CD235a/b+ population contains the majority of mesoderm cells that give rise to the hematopoietic fates. It is not necessary to remove the KDR+CD235a/b− cells.

The contacting of the BMPRA-Ri population of cells with a mesoderm specifying culture composition comprising a BMPR1/R2 agonist, an FGF receptor agonist and an activin receptor agonist allows for concurrent exposure of the population of cells with the indicated agonists. These agonists can be added to the culture composition prior to the culture composition being contacted with the BMPRA-Ri population of cells. Alternatively, a specifying culture composition lacking these agonists (and/or other components) can be contacted with the BMPRA-Ri population of cells and the agonists (or one or more of them) can be added subsequently.

In some embodiments, the PSCs are cultured, for example using one or more of the steps or reagents described in Example 1 for hPSC culture.

In an embodiment, the BMPRA-Ri population of cells; are contacted with the mesoderm specifying culture composition comprising a BMPR1/R2 agonist, an FGF receptor agonist and an activin receptor agonist for about 3 days, at least 3 days or up to 3 days. For example about 3 days may include contact for anywhere between 64 hours and 80 hours. At least 3 days may include at least 64 hours and up to 3 days may include up to 80 hours

The mesoderm specifying culture composition comprises a suitable base media such as StemPro-34 media, GMEM, IMDM, RPMI, STEMSpan SFEM, STEMdiff APEL2 and/or X-VIVO. Depending on the base media, one or more supplements may be added.

The pluripotent stem cells can be human pluripotent stem cells. The pluripotent stem cells can be embryonic cells or induced pluripotent stem cells (iPSCs) for example human induced pluripotent stem cells (hiPSCs).

In one embodiment, the pluripotent stem cells when contacted with the PSC culture composition and/or the BMPRA-Ri population of cells when contacted with the mesoderm specifying culture composition are in the form of embryoid bodies (EBs). The embryoid bodies can be obtained by culturing the pluripotent stem cells and/or the or the BMPRA-Ri population of cells to aggregate the cells.

For example as indicated in Example 1, embryoid bodies can be formed, by orbital shaking for about 18 hours in PSC culture composition comprising BMPRA and Ri prior to contacting the resulting population with the mesoderm specifying culture composition.

As described in the Examples, differentiation cultures can be maintained in hypoxic conditions for example cells such as the PSCs after formed as embyroid bodies were contacted with the mesoderm specifying culture composition at 37° C., 5% CO₂, 5% O₂.

Accordingly, in some embodiments, one or more of the culturing steps is performed under hypoxic conditions, optionally wherein the hypoxic condition is a cell culture incubator environment of 5% CO₂ and 5% O₂, and/or addition of a hypoxia inducible factor (HIF) prolyl-hydroxylase (PHD) inhibitor (HIF-PHDI). Various HIF-PHDIs are known. In one embodiment, the HIF-PHDI is a tricyclic triazole compound, optionally IOX2, IOX4, DMOG or similar compounds that also increase HIF1a signaling. In another embodiment, the HIF-PHDI is selected from Daprodustat, Molidustat, Roxadustat, Vadadustat and Desidustat.

When the method involves forming embryoid bodies, the first day of the protocol is forming embryoid bodies. The PSCs are cultured in PSC culture medium for about 16-24 hours, for example 18 hours to form the embryoid bodies (EBs) and the EBs (e.g. the BMPRA-Ri population of cells) are treated for about 3 days in mesoderm specifying culture composition after the formation of the embryoid bodies or what is referred to as day 4 of the differentiation protocol. About 3 days includes for example +/−10% days, for example 2.7 days to about 3.3 days.

As demonstrated herein the concurrent use of BMPR1/R2 agonist such as BMP4, FGF receptor agonist such as FGF2 and activin receptor agonist such as Activin A, produces KDR+mesoderm cells that were able to generate T lymphoid lineage cells. KDR mesoderm such as KDR+CD235a/b+ mesoderm cells and/or KDR+CD235a/b− mesoderm cells can be used.

The BMPRA or BMPR1/R2 agonist can be any molecule or combination that activates BMPR1/R2 signaling such as BMP4. Others include BMP2, and/or BMP7.

In one embodiment, the BMPRA or BMPR1/R2 agonist is or comprises BMP4. The concentration of BMP4 in the composition contacted with the BMPRA-Ri population of cells is from about 0.5-100 ng/mL or any 0.1 increment from 1.1 to 99.9 ng/m L, preferably from about 1 ng/mL to about 30 ng/mL. In one embodiment, the concentration is about 10 ng/mL of BMP4.

The FGF receptor agonist can be any molecule that activates FGF receptor such as FGF2.

The FGF receptor agonist is preferably FGF2 (also referred to as bFGF) but can be any FGF or FGF analog that promotes KDR+ e.g. KDR+CD235a/b+ specification. The FGFs receptor agonist when FGF2, can be provided at a concentration from about 0.5 ng/ml to about 100 ng/ml, or any 0.1 increment from 1.1 to 99.9 ng/mL, preferably from about 1 ng/mL to about 30 ng/mL, optionally at about 5ng/m L.

The activin receptor agonist can be any molecule that activates Nodal signaling through one of its receptors, ALK4, ALK7, ACTRIIA and/or ACTRIIB such as activin A or Nodal.

In one embodiment, the concentration of Activin A in the composition contacted with BMPRA-Ri population of cells is from about 0.2 ng/mL to about 100 ng/mL, for example about 1 ng/mL, 2 ng/mL, about 3 ng/mL, about 4 ng/mL, about 5 ng/mL, about 6 ng/mL, about 7 ng/mL, about 8 ng/mL, about 9 ng/mL or about 10 ng/mL. Higher concentrations can also be used, for example where the ratio of BMPR1/R2 agonist and FGF receptor agonist are maintained.

In some embodiments, the concentration of Activin A is greater than about 2 ng/mL and less than 8 ng/nL. The optimal concentration can be determined for example using a titration experiment, for example as described in the Examples. As an example, different amounts of Activin A (e.g., 0 to 10 ng/mL in 2 ng/mL increments) in the presence of a fixed concentration BMP4 (10 ng/mL) and FGF2 (5 ng/mL) is added to cultures on day 1 of differentiation. After about 3 days the percentage of KDR+CD235a/b+ cells is quantified. The optimal concentration is the condition that provides the highest frequency of KDR+CD235a/b+ cells. In addition or alternatively the proportion of CD43+ cells in cultures continued to day 9 of differentiation can be assessed. If different concentrations of Activin A result in the greater number of desired cells, either concentration or the lower concentration may be used.

In one embodiment, the ratio of BMPR1/R2 agonist, FGF receptor agonist and activin receptor agonist is about 10:5: 6 or about 10:5:2 or between about 10:5: 6 and 10:5:2. For example, the concentration of a particular agent can be that which provides similar effect to BMP4, FGF2 and Activin A used in a ratio of about 10:5:6 or about 10:5:2 or between about 10:5:6 and 10:5:2.

As mentioned, the pluripotent stem cells can be any pluripotent stem cell line or source or can be induced pluripotent stem cells. The inventors used an embryonic stem cell line and an induced pluripotent stem cells derived from a cells from a patient. Methods for making induced pluripotent stem cells (i.e. pluripotent stem cells artificially derived (e.g., induced or by complete reversal) from a non-pluripotent cell, typically an adult somatic cell) are known. For example, iPSC by inducing expression of one or more genes (including POU4F1/OCT4 (Gene ID; 5460) in combination with, but not restricted to, SOX2 (Gene ID; 6657), KLF4 (Gene ID; 9314), cMYC (Gene ID; 4609), NANOG (Gene ID; 79923), LIN28/LIN28A (Gene ID; 79727)). For example, this can be accomplished with viruses that express these genes, including, but not limited to lentiviruses and Sendai viruses. Methods for making induced pluripotent stem cells include methods disclosed in U.S. Pat. Nos. 7,682,828, 8,058,065, each incorporated herein by reference. Methods for making CRISPR edited iPS cells are also known. Commonly used cells are peripheral blood mononuclear cells and dermal fibroblasts, as these are accessible.

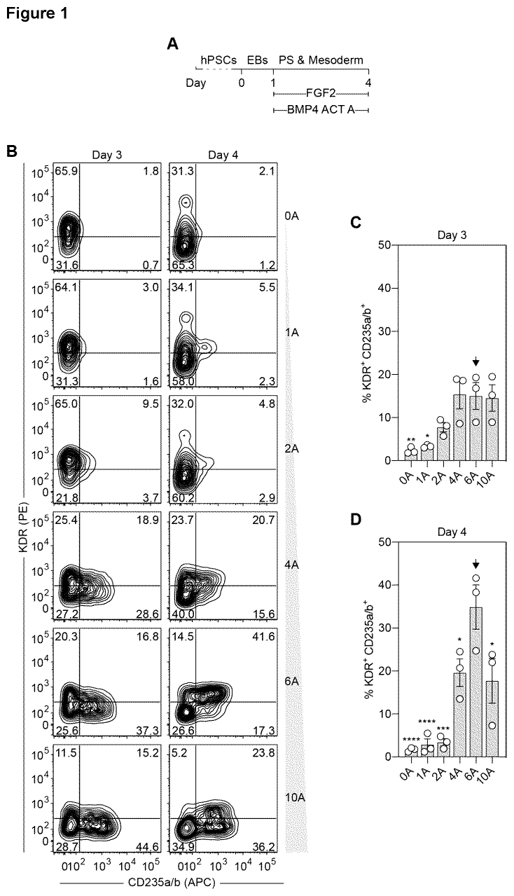

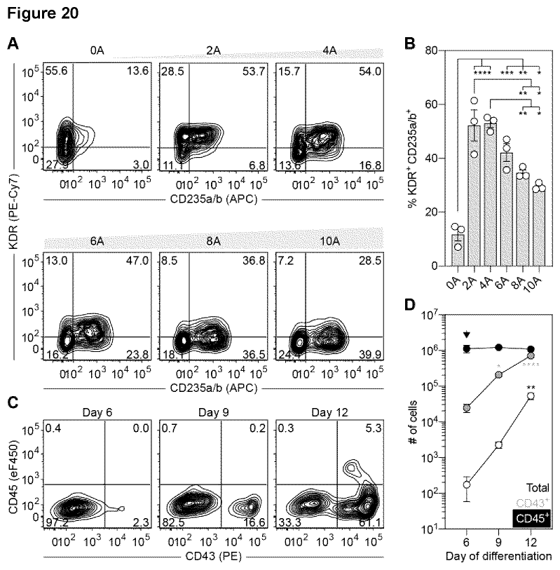

Referring to FIGS. 1 and 20, it is demonstrated that use of 6 ng/mL Activin A when the starting population was H1 ESCs or 2 ng/mL where the starting population was CHOPWT10 human iPSC line produced a population of cells where greater than 30% of the cells were KDR+CD235a/b+. Accordingly, in some embodiments, the concentration of activin receptor agonist used is a concentration or ratio that produces at least 30% KDR+ mesoderm eg. KDR+CD235a/b+ mesoderm cells after about 3 days of contact with the mesoderm specifying culture composition.

In some embodiments, the method further comprises contacting the KDR+ mesoderm, e.g. KDR+CD235a/b+ mesoderm cells with a HEC culture composition comprising VEGF, and optionally an FGF receptor agonist such as FGF2 and optionally hematopoietic cytokines such as IL-6 and IL-11 to obtain CD34+KDR+ hemogenic endothelial cells (HECs). As demonstrated herein, most of hematopoietic mesoderm is CD235 a/b+ but some CDR235a/b− cells are present. It is not necessary to remove CDR235a/b− cells when generating HECs.

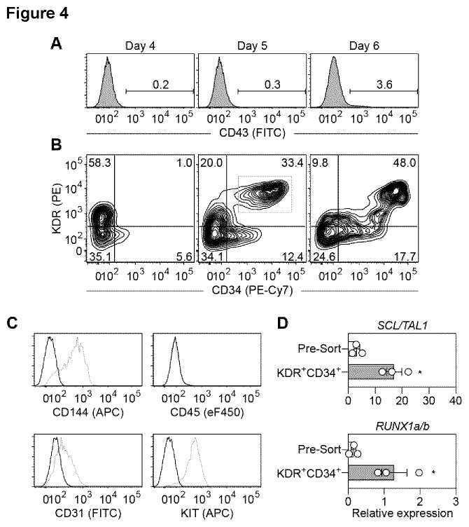

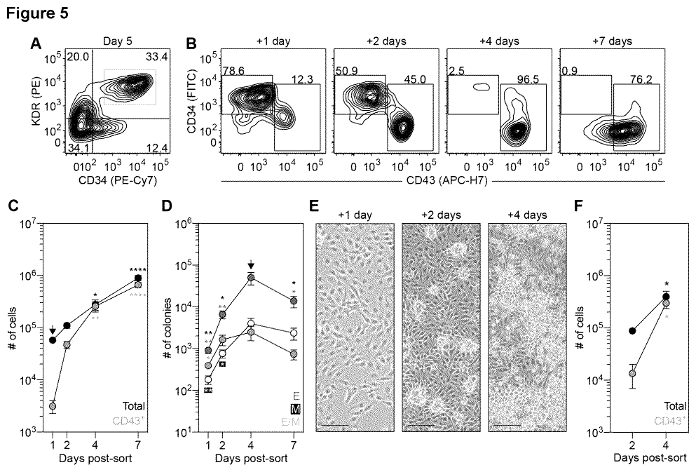

As demonstrated for example in FIGS. 4 and 5, the inventors demonstrate that KDR+CD235a/b+ mesoderm cells can be further differentiated to HECs which express for example KDR, CD34, KIT, CD144 and/or CD31.

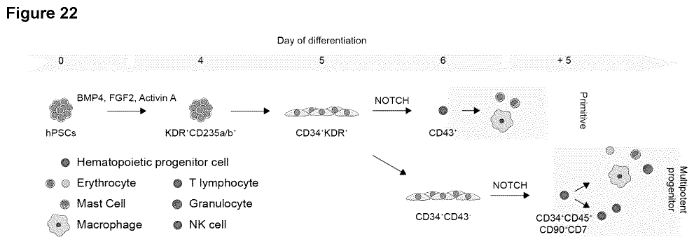

As demonstrated in the Examples, HECs appear at day 5 (about 1 day after KDR+CD235a/b+ mesoderm cells). The HECs include cells that will differentiate along the primitive pathway cycling through CD43+ hematopoietic progenitor cells which are detected starting at around day 6. Notch ligand present in HEC cells, for example when the cells are in embryoid bodies, can for example provide the Notch signal required to differentiate along this pathway and produce for example mast cells, macrophages and primitive type erythrocytes (see FIG. 22).

As demonstrated in the Examples, the CD34+KDR+ HECs can be cultured as aggregates providing a source of Notch agonist.

The HECs which are CD43- also include CD34+CD43− cells present around day 6 and which can develop along the multipotent progenitor pathway cycling through CD45+ hematopoietic progenitor cells. Day 6 CD34+CD43− HECs can be isolated and optionally aggregated, and cultured with VEGF and optionally an FGF receptor agonist and optionally one or more hematopoietic cytokines and in some embodiments an external Notch activation source to obtain for example CD34+CD45+CD90+ hematopoietic cells and eventually T lymphoid cells, NK cells, granulocytes, macrophages and multipotent type erythrocytes as further described herein.

In some embodiments, the KDR+CD235a/b+ mesoderm cells are contacted with the HEC culture composition comprising VEGF and optionally FGF receptor agonist and one or more hematopoietic cytokines for at least 0.5 days, 0.75 days or at least or about 1 day. The incubation is typically less than 2 days or 1.5 days and before day 6 of differentiation as blood progenitor cells of the primitive pathway appear on or around day 6. Primitive hematopoietic progenitors that appear on or around day 6 can be isolated based on the expression of CD43+. These progenitors as shown herein give rise to for example to erythroid, macrophage and mast cell lineages.

The HEC culture composition comprises a suitable base media such as StemPro-34 media into which VEGF and optionally FGF receptor agonist and one or more hematopoietic cytokines such as IL-6 and/or IL-11 can be added.

As demonstrated in the Examples, the HECs can be differentiated to provide primitive lineage hematopoietic progenitor cells and multipotent lineage hematopoietic progenitor cells.

For example, if primitive lineage progenitor cells are desired, the method can further comprise contacting the CD34+KDR+ HECs with a primitive progenitor culture composition comprising VEGF, and optionally an FGF receptor agonist and one or more hematopoietic cytokines such as IL-6, IL-11, SCF, IGF1 and/or EPO and providing a Notch agonist signal to obtain CD43+ hematopoietic progenitor cells. The primitive progenitor culture composition can be similar or the same as the HEC culture composition. The primitive progenitor culture may comprise hematopoietic factors, as IL-6, IL-11, SCF, IGF1 and/or EPO as well as others such as GM-CSF or M-CSF.

In one embodiment, the method comprises contacting pluripotent stem cells (PSCs) with a PSC culture composition comprising a BMP receptor agonist (BMPRA) and optionally a ROCK inhibitor (Ri) to produce a BMPRA-Ri population of cells, preferably wherein the PSCs are cultured with the PSC culture composition in a manner for forming EBs (this can be referred to as day 0-1); contacting the BMPRA-Ri population of cells with a mesoderm specifying culture composition comprising a BMPR1/R2 agonist, an FGF2 receptor agonist and an activin receptor agonist to produce KDR+CD235a/b+ mesoderm cells, preferably BMP4, FGF2 and Activin A (e.g. on days 1-4); contacting the KDR+CD235a/b+ mesoderm cells with a HEC culture composition comprising VEGF and optionally FGF receptor agonist such as FGF2 and optionally one or more hematopoietic cytokines, preferably comprising VEGF, FGF2, IL6, and IL-11, to obtain CD34+KDR+ hemogenic endothelial cells (HECs) (e.g. on days 4-6); and
i) expanding and/or differentiating the HEC cells in primitive progenitor culture medium comprising for example VEGF, FGF2, IL-6, IL-11, EPO, IGF1 and SCF; or
ii) isolating CD34+CD43− negative cells expanding and/or differentiating the HEC cells in multipotent progenitor (MPP) culture medium, optionally in the presence of a Notch ligand.

Steps i) and ii) produce hematopoietic progenitor cells that differentiate to different blood cells as shown for example in FIG. 22.

Referring to FIGS. 2 and 4, it is demonstrated that when cells were cultured in primitive progenitor culture composition, cells expressing CD43 emerged as early as day 6 of differentiation (e.g. where the CD34+KDR+ HECs had been in contact with the primitive progenitor culture composition for about 1 day). The number of such cells increased with time in culture for example at least until day 9 (see FIG. 2) and remain even after 15 day (e.g. after 11 days of treatment with hematopoietic cytokines). Progenitors may be present after 20 days or even after 24 days although their numbers may be low.

In some embodiments, the CD34+KDR+ HECs are contacted with the primitive progenitor culture composition comprising for example FGF2, VEGF, and hematopoietic cytokines such as IL-6 and IL-11, for at least 1 day, or about 2 days and subsequently FGF2, VEGF, and hematopoietic cytokines such as IL-6, IL-11, SCF, IGF1 and/or EPO for an additional 1 day, 2 days, 3 days, 4 days, 5 days, 6 days, 7 days, 8 days, 9 days, 10 days, 11 days or 12 days, or longer, for example until the desired number of CD43+ cells are obtained.

For example, CD7+ progenitor cells were generated after about 12 days of culturing day 6 CD34+CD43− HECs with a Notch source (e.g. OP9-DL4 cells) (e.g. 18 days from start with PSCs). CD7+ cells can be isolated using an anti-CD7 antibody and for example FACS. Similarly other cell types can be isolated based on the presence of cell surface markers.

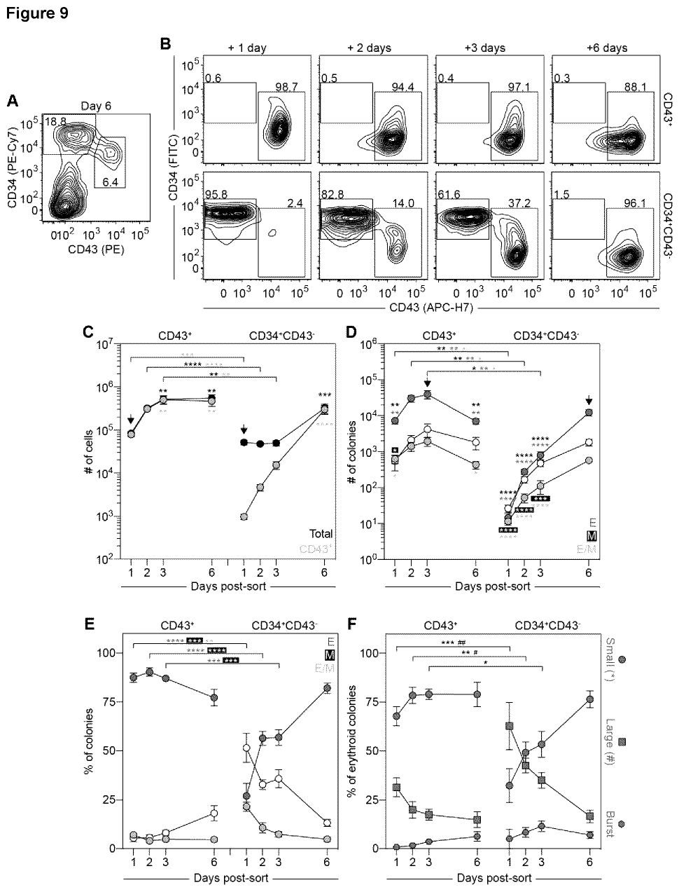

If greater number of progenitor cells are preferred, the contacting can be for example less than 4 days, less than 5 days or less than 6 days. If more mature cells are preferred the contacting can for example be more than 6 days. As shown for example in FIG. 9, CD43+ population increased overtime with the colony forming progenitors decreased between days 3 and 6 of culturing with VEGF, FGF2 and hematopoietic cytokines.

The primitive progenitor culture composition can comprise a suitable base media such as StemPro-34 media and comprising VEGF and optionally one or more of FGF2, and hematopoietic cytokines such as IL-6, IL-11, SCF, IGF1 and/or EPO.

As demonstrated in the Examples, contacting the CD34+KDR+ HECs gave rise to various lineages of the primitive program.

In one embodiment, the method further comprises culturing the CD43+ hematopoietic progenitor cells and generating macrophage cells. For example, as demonstrated in the Examples, the CD43+ hematopoietic progenitors can be differentiated to the macrophage fate using stage-specific factors. CD43⁺ cells are optionally isolated, for example on day 9 of differentiation and cultured in primitive progenitor media supplemented with MCSF, IL3 and SCF. GM-CSF can also be added. As demonstrated in the Examples, after about 3 days of culture, the cells were collected and cultured in StemPro-34 media supplemented with MCSF. The media was changed every 3 days for the remainder of the differentiation and cultured up to 40 days. Cultures were generally highly enriched in CD45⁺CD68⁺CD14⁺ macrophages after 13 days of culture (e.g. after adding MCSF).

Macrophages lineages can be differentiated from CD43+ hematopoietic progenitor cells isolated for example till any day including or after day 6, before day 15, for example day 6 or 9 or 12 (e.g. from PSCs).

For example CD43+ cells can be isolated at day 9 of differentiation and treated for example for about 3 days with SCF, IL3 and MCSF. After 3 days the cells can be removed and transferred to media only containing MCSF.

The primitive macrophage (also referred to as myeloid lineage) acquires CD45 expression, as the cells mature over a period of 4-6 days.

In one embodiment, the method further comprises culturing the CD43+ hematopoietic progenitor cells and generating mast lineage cells. The prognitors of the mast lineage cells begin to appear for example day 6. Mast cell inducing factors can be added such as IL3 and SCF to increase numbers of mast cells produced. In some embodiments, the mast cell lineages are isolated.

In another embodiment, the method further comprises culturing the CD43+ hematopoietic progenitor cells and generating primitive erythrocytes. Progenitors of the primitive erthyrocyte lineage cells begin to appear for example at day 6 of differentiation. Erythrocyte inducing factors can be added such as, EPO, SCF, IL3 and IGF1 to increase numbers of erythocyte cells produced. In some embodiments, the erythroid cell lineages are isolated. As demonstrated in the Examples, in addition to the CD43+ population that emerges on day 6 of differentiation, the inventors have identified CD45− population for example a CD34+CD43−CD45− population that co-expressed the HEC/endothelial cell markers KDR, KIT, CD144 and CD31 and can be used to generate cells of the multipotent progenitor lineage. Accordingly, in some embodiments, the method further comprises culturing the CD34+KDR+ HECs for a period of time in HEC culture media to produce CD34+CD43− cells. These HEC cells may be present in EBs or adherent cells.

Progenitors of the primitive pathway can be removed and/or day 6 CD34+CD43− cells can be isolated for example by FACs. When cells are in EBs or otherwise aggregated, cells are disaggregated prior to isolation by for example FACs optionally as described herein.

The period of time the HECs are cultured in HEC culture media can be about 1 day to about 2 days and cells can be isolated on what is referred to as day 6 of differentiation. The cells can be isolated for example by FACs or MACS, for example as described herein.

Once isolated, the CD34+CD43− HECs can be contacted with a multipotent progenitor culture composition comprising one or more hematopoietic cytokines such as IL-6, IL-11, SCF, IL-7, FLT3L, IGF1 and/or EPO and optionally a Notch agonist to obtain CD34+CD45+ hematopoietic progenitor cells, optionally CD34+CD45+CD90+CD7− and/or CD34+CD45+CD90−CD7+ hematopoietic progenitor cells.

In an embodiment, the method comprises isolating CD34⁺CD45⁺CD90⁺CD7⁻ cells. As described herein, CD34⁺CD45⁺CD90⁺CD7⁻ cells are multipotent hematopoietic progenitors with erythroid, myeloid, NK cell and T lymphoid potential.

Some embodiments, employ addition of a Notch agonist. In one embodiment, the Notch agonist is a Notch ligand (e.g. Delta-like (DL) and Jagged (Jag)), provided for example by a cell line expressing the ligand. Notch can be provided for example where the CD34+KDR+ HECs contacted with the multipotent progenitor culture composition are grown on a Notch expressing cell line such as OP9-DL4, OP9-DL1, OP9-JAG1, MS5-DL4, MS5-DL1 or MS5-JAG1 or a scaffold such as a culture plate or a bead comprising an adhered or otherwise immobilized Notch ligand. The Notch ligand may be bead-bound Notch ligand (e.g. DL4 ligand). Approaches using a scaffold immobilized Notch ligand do not require for example, serum or stromal cells.

For example, the cell lines can be made by transducing the cell line with a Notch ligand expressing virus. Kits comprising immobilized DL4 for making T cells are also available for example from Stem Cell Technologies.

CD43− HECs when contacted with a Notch ligand expressing cell can be contacted in MPP wherein the base media is alpha-MEM the hematopoietic cytokines are IL7, FLT3L and SCF and serum.

In some embodiments, the method further comprises isolating CD45+ cells. The CD34+CD45+ cells are or comprise hematopoietic progenitor cells of the MPP pathway.

The CD34+CD45+ hematopoietic cells can can be used to generate cells of the multipotent lineage as shown for example in FIG. 22.

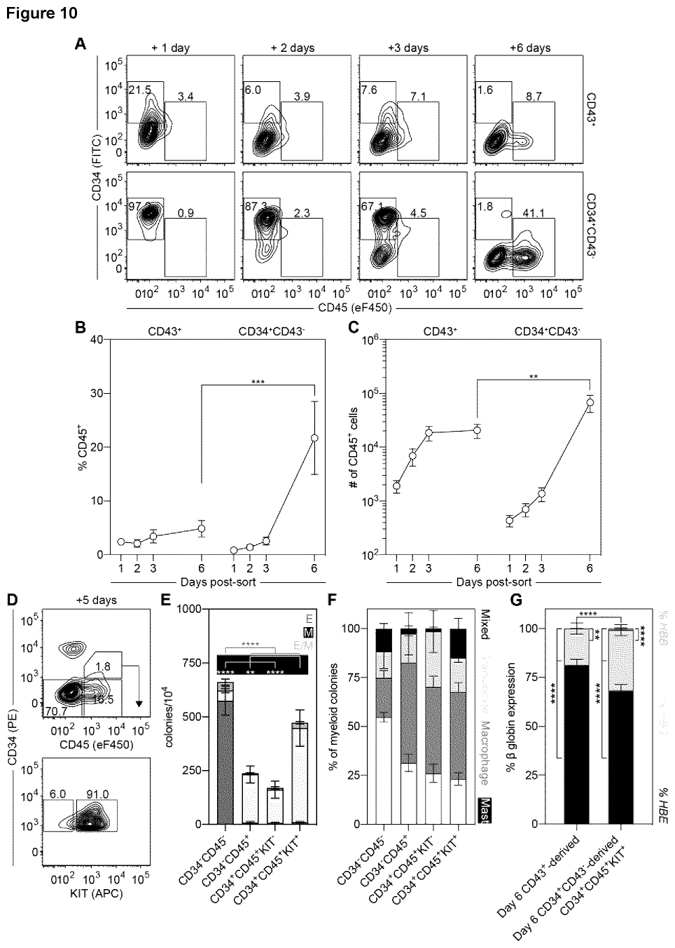

As shown in for example FIG. 10, culturing CD34+CD43−CD45− HECs in MPP with the indicated factors resulted in precursor cells for various lineage cells of the MPP pathway. In some embodiments, the method further comprises isolating CD34+CD45+KIT+, CD34+CD45+KIT−, CD34−CD45+ or CD34−CD45− cells. Said cells are detectable for example after about 3 days, 4 days or 5 days after culturing HECs in MPP in the presence of Notch ligand, for example provided by the culture of the HECs with OP9-DL4 cells.

In some embodiments, CD34+CD43− cells (e.g. day 6 of differentiation) are cultured for up to or about 5 days (e.g. 11 days of total culture) to obtain the MPP hematopoietic progenitor cells.

As demonstrated in the Examples, the method comprise contacting the CD34+CD45+ hematopoietic progenitor cells optionally the CD34+CD45+CD90+CD7− hematopoietic progenitor cells with the multipotent progenitor culture composition comprising one or more hematopoietic cytokines and optionally a Notch agonist to obtain multipotent lineage cells.

Various factors can further be added to direct differentiation towards are described cell type using for example factors described herein for macrophages, mast cells and erythrocytes.

The methods described herein can also be used to generate NK progenitor cells and NK mature cells. For example, the MPP culture composition can comprise one or more factors that promote NK differentiation such as IL7 and FLT3L. NK cells are detectable for example around 15 days of culture with OP9-DL4 cells.

The methods described herein can also be used to generate granulocyte progenitor cells and granulocyte mature cells. For example, the MPP culture composition can comprise one or more factors that promote granulocyte differentiation such as GCSF. Granulocyte progenitor cells are detectable for example after 2 days of isolating the day 6 CD34+CD43− HECs.

The methods described herein can also be used to generate T lymphoid progenitor cells and T lineage mature cells. For example, the MPP culture composition can comprise one or more factors that promote T cell differentiation such as IL7 and FLT3L. CD3+ T cells are detectable for example around day 25 of culture of the day 6 CD34+CD43− HECs with OP9-DL4 cells.

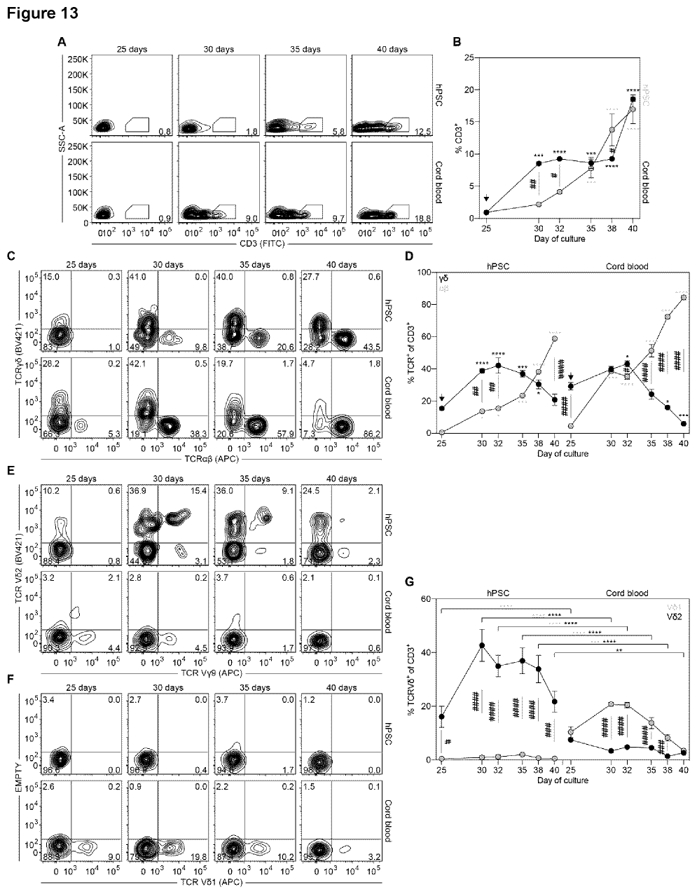

As demonstrated in the Examples, the methods can be used to produce particular subsets of T cells, expressing a desired TCR. Cultures that contain different subsets of TCR+ T cells is shown in FIG. 13.

In some embodiments, the method further comprises isolating one or more of the multipotent program lineage cells. For example a particular lineage can be isolated or a combination of lineages.

In one embodiment, macrophage lineage cells are isolated.

In one embodiment, mast lineage cells are isolated.

In one embodiment, multiopotent program lineage erythrocytes are isolated. As demonstrated herein, erythrocytes derived from CD34+CD43− HECs predominantly express, when assayed by PCR, fetal beta globin.

In one embodiment, granulocytes are isolated.

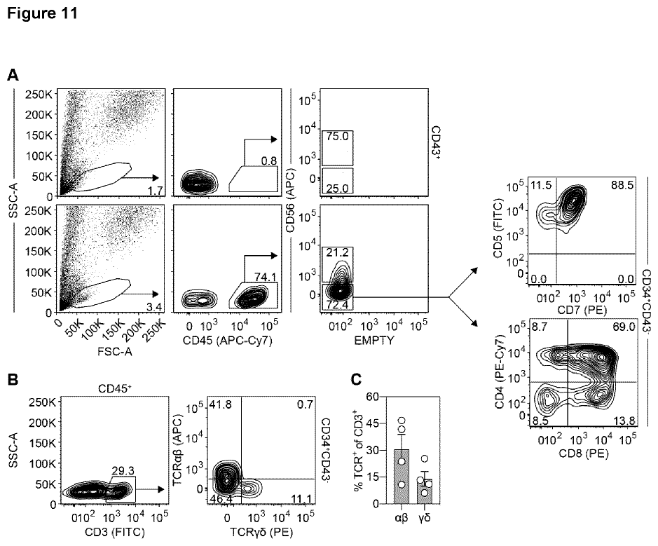

Referring to FIG. 11, the inventors demonstrate that the

CD34+CD43− population comprised T lymphoid lineage precursors which can be differentiated using a notch ligand for an extended period of time, for example at least or about 5 days. For example CD7 is an early marker of T lymphoid differentiation. This is seen at 5 days of culture with OP9-DL4 cells. Mature (CD3+) lymphocytes are seen for example by around 25 days. For example, Notch agonist receptor stimulation for 1 month produced T lymphoid progenitors and an additional 10 days of culturing produced αβ or γδ TCR+ lymphocytes Accordingly, In an embodiment, the method comprises extended culture of the CD34+CD43− HEC and optionally isolation of the T lymphoid lineage cells. The extended culture can be until the desire progenitor or mature cell population is obtained, for example at least 25 days, at least 30 days, at least 35 days, at least 40 days, at least 45 days or longer.

It is also demonstrated herein that T cells that expressed the Vδ2 TCR could be isolated from Notch ligand (eg. OP9-DL4) cultures initiated with the progenitors of the MPP program. In one embodiment, Vδ2 TCR T cells are isolated.

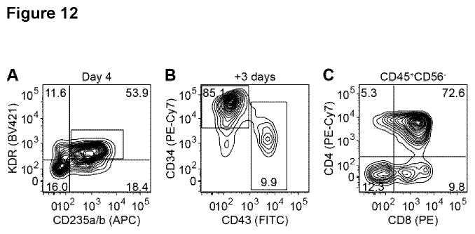

The KDR+CD235 a/b+ cells can be aggregated prior to exposure to Notch agonist receptor, which can as shown for example in FIG. 12 increase the frequency of CD34+CD43− cells.

In some embodiments, the Notch agonist is a Notch ligand.

The time of incubation can vary depend on various factors such as the starting population, concentrations of factors and the like. The times are given as examples. Emergence of the desired cell types can be monitored by assays such as FACs, PCR and the like and/or using a colony forming assay as described in the Examples.

The methods can also comprise isolating one or more of the primitive or MPP program lineage cells, optionally a population expressing a particular cell surface receptor or combination of receptors. These may define a lineage or multiple lineages depending on the desired cells to be isolated. Desired progenitors or mature cells (produced by either the primitive or the multipotent pathways) can also be isolated for example by FACs, magnetic activated cell sorting (MACS) and/or using affinity reagents

For example, erythroid progenitors can be isolated using affinity reagents for CD43 and/or CD235a/b; mast cell progenitors can be isolated using CD43, CD45 and/or KIT; macrophage progenitors can be isolated using CD43 and/or CD45 and for example CD31 and/or CD34; and megakaryocyte progenitors can be isolated using CD41.

Macrophages can be isolated by FACs, magnetic activated cell sorting (MACS) and/or using affinity reagents that target CD45⁺, CD68⁺, and/or CD14⁺. Examples for other mature cells are described herein.

As demonstrated in the Examples, cells at different stages of differentiation can be assayed by FACs, isolated optionally by FACS and/or affinity reagents using markers described herein or PCR. Examples of genes and primer sequences that can be used to confirm that the differentiation is progressing are provided in Table 1.1.

In some embodiments, one or more types of the isolated cells are resuspended in a composition. In some embodiments, particular cells are isolated for example cells expressing a particular marker or set of markers.

The cells described herein can be resuspended in a composition such as a gel such as a hydrogel, optionally in combination with another cell type such as cardiomyoctes or hepatocytes. The gel is preferably biocompatible.

The cells described herein can also be resuspended in a composition that is a sterile osmotically balanced fluid solution such as a saline solution (e.g. 0.9% saline) or other biocompatible fluid such as balanced cystalloid solutions (e.g. Plasma-Lyte) comprising sodium, potassium and chloride or a culture medium including one described herein, optionally a base medium and/or comprising additional components. Other solutions useful as freezing solutions can also be used and include for example CryoStro CS10 (animal-free freezing media from BioLife solutions cat #210102)

Details of method that can be used are provided in the Examples. One or more the steps that are described can be used as suitable in the methods described herein.

Cell comprising a particular cell surface marker can be isolated by Flourescence activated Cell Sorting (FACS) purification and/or using a marker specific affinity reagent. For example, the affinity reagent may be conjugated to beads. In some embodiments, the methods comprise one or multiple purification rounds for example one or more multiple FACS purifications or affinity beads based for example on cell surface expression patters described herein.

In one embodiment, the beads are magnetic beads for magnetic based separation of cells, optionally polystyrene spherical beads that are superparamagnetic.

In some embodiments, one or more enrichment steps are performed.

In one embodiment, the population is an isolated population and/or an in vitro produced population. For example, an aspect includes an in vitro produced cell according to a method described herein.

As described herein, the methods produced cell populations that are enriched for a particular cell type. In an embodiment, the methods involve producing a population comprises at least 30%, at least 35%, at least 40% or at lest 50%, of the desired cell type. In other embodiments cells can be isolated to provide for example greater numbers, for example at least 60%, at least 70%, at least 80% or at least 90% of cells expressing one or more markers described herein.

The various components described herein can be added to the media daily and/or the media comprising the component can be replenished daily. In other embodiments, the component(s) or media comprising the component(s) is added to the cells every 2 days or only once during the particular culture period, for example up to 6 days.

For example, the addition of some components can be by direct addition to the cells in media or by replacing the media with new media containing components. Components for some steps can for example be replaced daily from day 1-3 and replaced every 2 days from day 4 or 6 onwards. Typically, the mesoderm specifying media comprising BMPR1/R2 agonist, FGF receptor agonist and activin receptor agonist is added to KDR+CD235a/b+ on day 1 and not changed during the specifying step (e.g. days 1 to 3).

The method may involve for example removing all media and replacing with media comprising the particular components depending on the stage of differentation.

Another aspect is a population of cells comprising one or more types of the cells produced according to a method described herein. In one embodiment, the population is isolated after a method step described herein. For example, the CD34+CD7+ T lymphoid progenitor population cells that are as generated using a method described herein are initially CD5 negative unlike T lymphoid lineage cells generated from cord blood.

In an embodiment, the population of cells comprises CD34⁺CD45⁺CD90⁺CD7 hematopoietic progenitor cells.

In an embodiment, the population of cells comprises CD45⁺CD56⁻CD4⁺CD8⁺ cells.

In another embodiment, the population of cells comprises Vδ2 T cells.

In an embodiment, the population of cells is a purified population. In an embodiment, the population of cells is not isolated from peripheral blood. In an embodiment, the population of cells is produced from iPSCs. In another embodiment, the population of cells comprises at least 1×10⁷ cells. In an embodiment, the population of cells are human cells.

As discussed herein it has not been easily possible to isolate Vδ2 T cells as they are rare in the adult. The present method provides methods and compositions comprising for example at least 1×10⁷ Vδ2 T cells.

In an embodiment, the population of cells is a purified population of cells comprising at least 80%, 85%, 90%, 95%, 98%, 99% or more of a particular population, such as CD34⁺CD45⁺CD90⁺CD7 hematopoietic progenitor cells.

A further aspect is a composition comprising one or more types of the cells (e.g the population of cells) described and/or isolated cells (e.g. isolated population of cells) and optionally a carrier or diluent.

Suitable diluent includes for example a suitable culture medium including for example medias such as base medias and medias comprising one or more components (eg. One or more factors added to a media) described herein, or freezing medium containing for example serum, a serum substitute or serum supplement and/or a suitable cryoprotectant such as dimethyl sulphoxide (DMSO), glycerol Methylcellulose or polyvinyl pyrrolidone. In some embodiments the diluents are sterile.

In one embodiment, the carrier is a pharmaceutically acceptable carrier.

In an embodiment, the composition is a gel such as a hydrogel and the cells are comprised in or on the gel. The gel is preferably biocompatible. In some embodiments, the composition is a liquid. For example, the composition may comprise a cell suitable diluent, optionally a cell media or other osmotically balanced nutrient solution. The cells and population of cells described herein and/or produced using the methods described herein can be combined in a composition in combination with another cell type such as cardiomyoctes or hepatocytes. The cells for example can be comprised in a pouch or comprised on a scaffold.

The in vitro produced cell or population of cells may be comprised in a vial such as a sterile vial.

In certain embodiments, the composition comprises a gel.

The composition can also comprise an osmotically balanced fluid solution.

In some embodiments, the composition is sterile. For example, cells can be grown under sterile conditions and resuspended in a solution or gel that is sterile.

The composition can be for administration to a subject in need thereof.

Another aspect is a cell implant composition comprising a gel, and one or more types of the isolated cells described herein, optionally in combination with cardiomyocytes or heptacytes.

A further aspect is a mesoderm specifying culture additive comprising:
a BMPR1/R2 agonist,
an FGF receptor agonist and
an activin a receptor agonist.

In one embodiment, the BMPR1/R2 agonist is BMP4.

In one embodiment, the FGF receptor agonist is FGF2.

In one embodiment, the activin receptor agonist is Activin A, optionally human Activin A.

In another embodiment, the activin receptor agonist is Nodal, preferably human Nodal.

In another embodiment, the amount of: the BMP4 is sufficient to provide within 0.5 ng/m Ito about 100 ng/ml, the FGF2 is sufficient to provide within 0.5 ng/ml to about 100 ng/ml and the Activin A is sufficient to provide within 0.5 ng/ml and 100 ng/ml, in a solution of about 500 mL, preferably wherein the ratio of the BMP4, FGF2 and Act A is about 10:5:6 or about 10:5:2 or between about 10:5:6 and about 10:5:2.

A mesoderm specifying culture composition comprising:
a hematopoetic progenitor suitable base media,
a BMPR1/R2 agonist,
an FGF receptor agonist, and
an activin receptor agonist.

The mesoderm specifying culture additive or compositions described herein can be for use in a method or kit described herein.

Also provided is a kit comprising a cell, cell population, additive or composition described herein or a vial comprising said cell or population of cells, one or more inducing or specifying components for producing cells described herein and/or instructions for producing one or more cells described herein.

The kit may include for example one or more of cells produced herein, optionally in freezing media and packaged in a coolant such as dry ice or liquid nitrogen, matrigel or equivalent ECM coated plate, basal growth media, one or more components described herein (e.g. FGF2, Activin A and BMP4), isolation antibodies and/or quantification antibodies, for example for use in FACs.

The methods, compositions additives and kits described herein can be used to produce said progenitor populations that would otherwise be unattainable, to further interrogate the function of tissue-resident immune cells.

### III. APPLICATIONS AND USES

In vitro and in vivo uses are also provided in other aspects.

In vitro produced cells described herein can be used in a screening assay to pre-screen candidate drugs.

Accordingly one aspect is a screening assay comprising
contacting a cell population produced according to a method described herein with a test agent;
measuring a desired read out, such as toxicity, of the test agent compared to the cell population treated with a vehicle control.

The in vitro cells can be used for example for testing drug candidates for human drug toxicity on pure or pooled human PSC derived backgrounds allowing ethnicity specific pharmacology testing and prevention of adverse drug reaction; and/or for testing and optimization of biological drug candidates (e.g. monoclonal antibodies, cytokines, small molecules etc.) for binding and clearance characteristics and improved biodistribution and circulation half-life.

In some embodiments, the in vitro produced cells are produced from human starting cells comprising a marker, such as a fluorescent marker, light emitting marker etc for tracking the human in vitro produced cells in an animal model. The animal is for example a rodent such as a mouse or a rat. For example, green fluorescent protein (GFP), or similar proteins, such as enhanced GFP (eGFP), RFP, CFP) or luciferase (Luc) can introduced into the human cells for tracking in the animal.

The progenitor cells can be used to provide tissue resident macrophages.

Protocols to generate microglia from hPSCs through the culture of macrophage progenitors with cells of the nervous system, such as astrocytes (Haenseler et al., 2017; Pandya et al., 2017) or in the presence of cytokines and growth factors, such as IL34 and TGFβ, which are known to be expressed in the brain (Abud et al., 2017; Douvaras et al., 2017; Muffat et al., 2016) exist. Accordingly the macrophage progenitors described herein can be used to generate microglia. In an embodiment, the method further comprises contacting a macrophage progenitor cell with a nervous system cell, optionally an astrocyte in a neural culture media comprising neural growth factors and/or cytokines such as IL-34 and/or TGFβ. Another aspect is use of a macrophage progenitor prepared and isolated as described herein e.g. isolated from a population of cells described herein, for example isolated using CD43 and/or CD45 and for example CD31 and/or CD34, to generate a microglia cell.

A similar approach can be undertaken to generate other populations of tissue-resident macrophages, for example, culturing macrophage progenitors with cardiomyocytes, cardiac fibroblasts/epicardium, and/or endocardium may be used to induce differentiation cardiac macrophages and to identify the regulatory mechanisms that guide their specification.

The methods described herein can also be used to make yolk sac like T cells. The multipotent progenitor program gives rise to T cells. Studies in the mouse have demonstrated that the yolk sac-derived T cells uniquely give rise to specialized T cell populations that reside in the skin throughout life. Likewise, a population of T cells (Vδ2) are abundant in human fetal development. The differentiation protocols described herein provide a source of these cells. The Vδ2 population could be isolated from for example Notch ligand e.g OP9-DL4, cultures initiated with the progenitors of the MPP program and cryopreserved for future transplantation or modification with for example CARs prior to use. Transformed Vδ2 give rise to cutaneous T cell lymphomas, which are highly aggressive, and no curative therapies exist. The transplantation of hPSC-derived Vδ2 may provide curative therapy through the replacement of the malignant cells (with or without allogenic HSC transplantation, chemotherapy or chimeric antigen receptor modification). These cells also have anti-microbial activity and can be used to treat antibiotic resistant infections. In addition, T-regulatory cells are abundant in fetal life. The hPSC-derived T cells may be an enriched source of these cells that could be used to induce tolerance following transplantation.

Accordingly also provided are use of cells (eg. population, mature or subsets such as Vδ2+ T lymphocytes) prepared using a method described herein as well as additives and compositions comprising additive and/or cells for preparing a medicament for transplantation, for treating cancer, for treating immunodeficiency (e.g. T cell immunodeficiency) or for treating antibiotic resistant infections and the like.

Studies in the mouse demonstrated that Vγ3+ T lymphocytes emerge early in development and contribute to the dendritic epidermal T cell (DETC) population in the adult skin. This cell population persists throughout life, with minimal contribution from HSC-derived progeny (Gentek et al., 2018b; Havran and Allison, 1988, 1990). The demonstration that the mouse yolk sac gives rise to Vγ3+ T lymphocytes following culture with OP9-DL1 cells (Yoshimoto et al., 2012) suggests that this lineage is generated as part of the LMP program. In human development, Vγ9+Vδ2+ T lymphocytes dominate the T cell repertoire at early stages of development (Dimova et al., 2015; Haynes and Heinly, 1995; Haynes et al., 1988; McVay and Carding, 1996) indicating that they represent the equivalent of mouse Vγ3+ cells. The inventors herein were able to show that the MPP population gives rise to both γδ and αβ T lymphocytes, which is consistent with potential of the E9.5 mouse yolk sac (Yoshimoto et al., 2012). As mentioned, the inventors were able to isolate Vδ2+ T cells (e.g. CD45+CD3+Vδ2).

Accordingly, the methods can be used to produce and use mature T cells or specific subsets thereof.

In an embodiment, the mature T cells or specific subsets are γδ T cells. In another embodiment, the mature T cells or specific subsets are CD45+CD3+TCRαβ+ cells. Other mature T cells and subsets are also contemplated as discussed herein.

Also provided in another aspect is a cellular therapy. In vitro produced cells can be produced and prepared as pharmaceutical compositions.

In some embodiments, the hPSC-derived T lineage cells can be used to treat immunodeficiencies, certain kinds of lymphomas (e.g. Vδ2 primary cutaneous gamma-delta T cell lymphomas are particularly aggressive and these cells may not be replaced by HSCs after transplantation). T cell lineage cells such as Vδ2 cells or CD45+CD3+TCRαβ+ cells may also be useful for preparing CAR-T cells. For example, gamma-delta T cells may function faster that alpha-beta T cells, which are normally used in CAR-T cell therapy for leukemia/solid tumors.

It is also showed herein that MPP-derived CD7+CD34+ T lymphoid progenitors differ in their expression of CD5 from cord blood-derived cells possibly identifying the earliest stage that the yolk sac and HSC-derived T lymphoid lineages diverge.

In one embodiment, the method comprises introducing a population of cells produced in vitro according to a method described herein, or a composition comprising said cells, into a subject in need thereof. In one embodiment, the population of cells or composition is introduced into the subject by injection. In another embodiment, the population of cells or composition is comprised along with cardiomyocytes and/or liver cells such as hepatocytes in a cell implant device, such as an immunoisolation device, vascular engraftment device, or multi cellular transplantation device, such as Encaptra® cell delivery system by ViaCyte.

Accordingly, also included is a method of providing a subject with progenitor cells, the method comprising administering a population of cells described herein, a composition comprising said cells or a cell implant comprising said cells to a subject in need thereof.

Various diseases and conditions can be treated.

A landmark study that specifically induced a BRAF mutation in Csf1r-expressing cells at E8.5 showed that dysfunctional yolk sac progenitors can cause disease in microglia, as these mice developed neurodegenerative disease (Mass et al., 2017). Because the yolk sac hematopoietic progenitors give rise to tissue-resident immune cells, it is possible that other diseases are also caused by mutations in these embryonic progenitors. Analyses of hematopoietic differentiation of iPSCs generated from individuals with trisomy 21 showed enhanced primitive erythropoiesis, at the expense of the megakaryocytic and myeloid lineages (Chou et al., 2012). Likewise, the frequency of megakaryocyte colony-forming progenitors was increased in EMP-like populations generated from trisomy 21 iPSCs (Maclean et al., 2012). These studies indicate that dysregulated hematopoietic cells derived from the yolk sac programs may be responsible for pediatric cases of leukemia in individuals with Down syndrome. Furthermore, the demonstration that hPSCs with the ETV6-RUNX1 fusion, which is common to pediatric B cell leukemias (Shurtleff et al., 1995), fail to complete B cell development (Boiers et al., 2018) suggests that a subset of these leukemias may also have a yolk sac progenitor origin. The onset of hematologic malignancies that develop from the progeny of the yolk sac programs may not be restricted to the pediatric setting. Primary cutaneous γδ T cell lymphomas (PCGDTLs) are an aggressive class of adult lymphomas that are derived from the γδ T lymphoid lineage (reviewed in Tripodo et al., 2009). Of the distinct subtypes of PCGDTLs, the panniculitic subtype is generated from malignant Vδ2+ cells and has a worse prognosis than other PCGDTLs, such as those initiated in Vδ1+ cells (Daniels et al., 2020). Because Vδ2+ T lymphocytes are specified at early stages of human development (Haynes and Heinly, 1995; Haynes et al., 1988; McVay and Carding, 1996), it is possible that mutations in yolk sac-derived progenitors accumulate over the life of the human and eventually cause this cancer. Analyses of the T lymphoid lineage that is generated from the MPP program may facilitate the identification of regulatory mechanisms that would render the malignant cells susceptible to therapy.

Accordingly, in another aspect the cells described herein provide models for assessing disease. For example, putative lymphoma-initiating mutations that have been identified in genomics studies could be engineered in wild type hPSCs to better describe how additional mutations are accumulated as these lymphomas develop. Alternatively, PCGDTL patient-derived iPSCs could be differentiated, as these cells would already harbour all of the genetic mutations of the lymphoma in the patient. The MPP-derived T lymphoid cells may also have therapeutic potential, as Vδ2+ T lymphocytes isolated from the peripheral blood of adults are able to efficiently kill different types of cancer cells in vitro and in vivo (reviewed in Hoeres et al., 2018).

The methods described herein can be used to make yolk sac like erythroid cells for transplanting to a subject in need thereof. The second yolk sac hematopoietic program (referred to as the EMP program in the mouse and now defined as the yolk sac multipotent progenitor program in the human) gives rise to an erythroid lineage that expresses the embryonic and fetal, but not adult β globin.

The methods described herein can also be used to make yolk sac like macrophages. The two yolk sac hematopoietic programs (primitive and yolk sac multipotent progenitor program) give rise to macrophages that seed the tissues and persist throughout life. In the mouse, these macrophages are essential for a reparative response to myocardial infarction (MI). The transplantation of hPSC-derived macrophages into the heart (with or without cardiomyocytes) may benefit patients that have experienced a MI or heart failure. Macrophages are known to remodel their environment indicating that the co-transplantation of macrophages with other cells (hPSC-derived or not) may facilitate engraftment. Macrophages may also prove beneficial for patients with liver fibrosis, through their ability to remodel fibrotic tissue.

The broad distribution of yolk sac-derived macrophages in the body also indicates that the transplantation hPSC-derived macrophages that have been modified could be used to deliver cargo (mRNAs, miRNAs, cytokines, etc.) to different tissues. These macrophages could also be engineered to recognize and destroy malignant cells (eg. with chimeric antigen receptors).This ability has been demonstrated in two models of solid organ cancers using macrophages differentiated from monocytes isolated from peripheral blood (Klichinsky et al., 2020). Given that the progeny of the yolk sac (primitive and MPP programs) are a major source of tissue-resident macrophages, it is possible that hPSC-derived macrophages would also home to the tissue and destroy the malignant cells. The hPSC-derived macrophages may also be used to remediate liver fibrosis, as the yolk sac is a potent source of Kupffer cells. This is predicted from preclinical studies and an ongoing clinical trial that uses macrophages differentiated from monocytes isolated from the peripheral blood (Haideri et al., 2017; Moroni et al., 2019).

Accordingly in one embodiment, macrophage cells as generated using methods described herein and/or their progenitors are administered to a subject suffering or who has suffered a cardiac infarction or a subject with liver fibrosis. For cell therapy embodiments, a cell population, composition or implant described herein may be administered.

The methods described herein can also be used to make yolk sac like NK cells. The multipotent progenitor population gives rise to NK cells. Lineage tracing in the mouse has shown that cells contained within the mouse yolk sac give rise to hepatic NK cells at later stages of development, which may represent the tissue-resident NK cells that persist in the liver throughout life. Transplantation of yolk sac-derived NK cells help induce tolerance following solid-organ transplantation or serve as a therapy to treat different kinds of malignancies (with or without chimeric antigen receptor modification). Although preliminary, chimeric antigen receptor (CAR)-modified NK cells isolated from peripheral blood are being studied to treat CD19+ leukemias (Liu et al., 2020). hPSC-derived NK cells are also under active investigation to treat solid tumors (NCT0384110).

Accordingly one aspect includes using NK cells or NK progenitors as generated using a method described herein to prepare a CAR modified NK cell.

Accordingly one aspect includes using NK cells or NK progenitors as generated using a method described herein to treat a solid tumor, for example by administering cells or a composition or cell implant comprising said cells to a subject in need thereof.

Derivatives of the yolk sac hematopoietic programs lead to several diseases, but the loss of these populations can also has negative consequences. Clonal hematopoiesis of indeterminant potential (CHIP) is the result of the progressive dominance of a hematopoietic clone in an individual that lacks other hematologic abnormalities (reviewed in Jaiswal and Ebert, 2019). While CHIP is associated with the risk of future hematologic malignancy, individuals with CHIP are also predisposed to develop cardiovascular disease. The dominant hematopoietic clone in individuals with CHIP commonly has mutations in TET2 (Genovese et al., 2014; Jaiswal et al., 2014). Interestingly, bone marrow transplantation of mouse Tet2-deficient donor cells increased atherosclerotic lesions in the aorta in recipients (Jaiswal et al., 2017) indicating that HSC derivatives promote disease. Analyses of the macrophages in the hearts of recipient mice revealed that the Tet2-null macrophages expressed cytokines associated with inflammation, including II1b and II6 (Jaiswal et al., 2017). The accumulation of mutant HSC-derived macrophages may be at the expense of the yolk sac-derived tissue-resident cardiac macrophage population that normally maintains tissue homeostasis. This raises the possibility that the transplantation of macrophages generated from the human primitive and MPP programs, systemically or directly into the heart could help minimize cardiovascular disease risk in individuals with CHIP by dampening the pro-inflammatory response of the mutant macrophages.

The cell therapy can also be a supportive cell therapy. For example, the cells described herein can be administered with cardiomyocytes.

The transplantation of yolk sac-derived macrophages may also benefit patients with other cardiac pathologies. These cells have therapeutic potential, as indicated by the demonstration that yolk sac-derived cardiac macrophages are required for myocardial regeneration in the neonatal mouse heart after injury (Aurora et al., 2014).

This lineage has also been shown to benefit the adult, as it limits the fibrosis that follows myocardial infarction (MI) (Dick et al., 2019; Lavine et al., 2014). This suggests that the transplantation of hPSC-derived macrophages with or without cardiomyocytes may improve outcomes in patients following an MI. Preclinical testing of the primitive and MPP-derived macrophages can be carried in rodent models of myocardial infarction for example LAD ligation, cryoinjury, etc. or established models of myocardial infarction in the rodent

The applications of yolk sac-derived macrophages are not limited to the heart. Studies using a mouse model of liver disease showed that the transplantation of hESC-derived macrophages significantly reduces fibrosis and activates the hepatic regenerative response (Haideri et al., 2017). These findings have led to a clinical trial in patients with liver cirrhosis using macrophages differentiated from peripheral blood monocytes (Moroni et al., 2019).

As mentioned access to yolk sac-derived macrophages also offers the opportunity to establish novel disease models including novel animal models. This has been pioneered in the brain, in studies in which where hPSC-derived microglia are transplanted into the brains of immunodeficient mice that express the human MCSF protein to model Alzheimer's disease (Hasselmann et al., 2019; Mancuso et al., 2019). Preclinical testing in these animals, or similar ones is essential to test the therapeutic potential of macrophages. The low affinity of mouse MCSF ligand for the human MCSF receptor, results in poor engraftment of macrophages in standard NSG mice. Therefore, specialized animals or cells that express human MCSF are required to test these questions.

### IV. EXAMPLES

### Example

### Methods

### hPSC Culture

The H1 human embryonic stem cell (hESC) line (Thomson et al., 1998) and CHOPWT10 human iPSC line (Maguire et al., 2016) were used in this study. hPSCs were maintained on irradiated mouse embryonic fibroblasts (iMEFs) in hESC media containing DMEM/F12 (Cellgro) with penicillin/streptomycin (1%, ThermoFisher), L-glutamine (2 mM, ThermoFisher), non-essential amino acids (1×, ThermoFisher), β-mercaptoethanol (55 μM, ThermoFisher) and KnockOut serum replacement (20%, ThermoFisher) on 0.1% gelatin (Millipore Sigma)-coated tissue culture plates. Media supplemented with FGF2 (H1: 15 ng/mL and CHOPWT10: 10 ng/mL, R&D Systems) was changed daily for 7 days prior to differentiation. hPSCs were routinely tested for mycoplasma. hPSCs were maintained in normoxic conditions (37° C., 5% CO2).

### Hematopoietic Differentiation from hPSCs

hPSC differentiation to the hematopoietic lineage was performed in StemPro-34 media supplemented with penicillin/streptomycin (1%, ThermoFisher), L-glutamine (2 mM, ThermoFisher), ascorbic acid (50 μg/mL, Millipore Sigma), transferrin (150 μg/mL, ROCHE), monothioglycerol (50 μg/mL, Millipore Sigma) and other stage-specific factors for example as described below. Differentiation cultures were maintained in hypoxic conditions (37° C., 5% CO₂, 5% O₂) unless otherwise indicated. On day 0, hPSC cultures at 80-90% confluency were treated with TrypLE for 3 minutes at 37° C. Thereafter, the 80-90% of the TrypLE was aspirated and the cultures were incubated at 37° C. for an additional 2 minutes. Small clusters of hPSCs (<5 cells per cluster) were generated by gentle pipetting and transferred to 4 mL of StemPro-34 media (Gibco) containing ROCK inhibitor Y-27632 (10 μM) and BMP4 (1 ng/mL) at 500,000 cells/mL. Embryoid bodies (EBs) were generated in 60 mm Petri dishes on an orbital shaker (H1: 70 RPM; CHOPWT10: 60 RPM) for 18 hours. On day 1 of differentiation, the EBs were collected by centrifugation at 40 RCF for 5 minutes and cultured in StemPro-34 media supplemented with BMP4 (10 ng/mL), FGF2 (5 ng/mL) and Activin A (H1: 6 ng/mL; CHOPWT10: 2 ng/mL). Cultures were maintained under static conditions in 5% poly(2-hydroxyethyl methacrylate) (Millipore Sigma)-treated tissue culture plates for the duration of the differentiation. On day 4 of differentiation (e.g. after 3 days in culture with mesoderm specifying culture composition), the EBs were collected by centrifugation at 150 RCF for 5 minutes and cultured in StemPro-34 media supplemented with FGF2 (5 ng/mL), VEGF (15 ng/mL), IL6 (10 ng/mL) and IL11 (10 ng/mL). From day 6 of differentiation onward, cultures were maintained in StemPro-34 media containing FGF2 (5 ng/mL), VEGF (15 ng/mL), IL6 (10 ng/mL) and IL11 (10 ng/mL), SCF (100 ng/mL), IGF1 (50 ng/mL) and EPO (4 U/mL). Thereafter, the media was replenished every 2 days for the duration of the differentiation. To induce the definitive program, day 1 EBs were collected by centrifugation at 40 RCF for 5 minutes and were cultured in StemPro-34 media supplemented with BMP4 (10 ng/mL), FGF2 (5 ng/mL). On day 2 of differentiation, the EBs were collected by centrifugation at 150 RCF for 5 minutes and cultured in StemPro-34 media supplemented BMP4 (10 ng/mL), FGF2 (5 ng/mL) and SB431542 (6 μM, TOCRIS). On day 3 of differentiation the EBs were again collected by centrifugation at 150 RCF for 5 minutes and cultured in StemPro-34 media supplemented with FGF2 (5 ng/mL), VEGF (15 ng/mL), IL6 (10 ng/mL) and IL11 (10 ng/mL). The cultures were maintained from day 6 of differentiation onward, as described above. All recombinant factors are human and were purchased from R&D Systems.

### Macrophage Differentiation

CD43⁺ hematopoietic progenitor differentiation to the macrophage fate was performed in 25% StemPro-34 media supplemented with penicillin/streptomycin (1%, ThermoFisher), L-glutamine (2 mM, ThermoFisher), ascorbic acid (50 μg/mL, Millipore Sigma), transferrin (150 μg/mL, ROCHE), monothioglycerol (50 μg/mL, Millipore Sigma) and other stage-specific factors. The StemPro-34 base media was diluted in IMDM prior to the addition of the supplements. Macrophage differentiation cultures were maintained in normoxic conditions (37° C., 5% CO₂) unless otherwise indicated. Cultures were maintained under static conditions in 5% poly(2-hydroxyethyl methacrylate) (Millipore Sigma)-treated tissue culture plates for the duration of the differentiation. 2,000,000 CD43⁺ cells isolated on day 9 of differentiation were cultured in 25% Stem Pro-34 media supplemented with MCSF (30 ng/mL), IL3 (50 ng/mL) and SCF (100 ng/mL). After 3 days of culture, the cells were collected by centrifugation at 200 RCF for 5 minutes and cultured in StemPro-34 media supplemented with MCSF. The media was changed every 3 days for the remainder of the differentiation and cultured up to 40 days prior to their use in downstream assays. Cultures were generally highly enriched in CD45⁺CD68⁺CD14⁺ macrophages after 13 days of culture.

### Flow Cytometry and Cell Sorting

EBs prior to day 9 of differentiation, monolayer cultures and cultures aggregated after cell sorting were dissociated with Trypsin (Corning) for 5 minutes at 37° C. From day 9 of differentiation onward, EB cultures were dissociated with Trypsin for 5 minutes at 37° C. prior to incubation in collagenase type II (0.2%, Worthington) for 1 hour at 37° C. Cells were stained at a concentration less than or equal to 5,000,000 cells/mL for 30 minutes at 4° C. in the dark. For flow cytometry, cells were stained in IMDM (Gibco) supplemented with penicillin/streptomycin (1%), FCS (2%, Wisent) and DNaseI (Millipore Sigma). For fluorescence-activated cell sorting (FACS), cells were stained, sorted and collected in StemPro-34 media supplemented with penicillin/streptomycin (1%), L-glutamine (2 mM) and DNaseI. Cells were sorted through a 100 μm nozzle using the Influx (BD), Ariall (BD), AriaIII (BD) and MoFlo (Beckman-Coulter) cell sorters at the SickKids/UHN Flow Cytometry Facility. Thereafter, the sorted cells were counted and resuspended at 250,000 cells/mL in StemPro-34 media supplemented with recombinant factors appropriate for the day of differentiation. To aggregate the sorted cells, 250 μL of the single cell suspension was transferred to individual wells of 5% poly(2-hydroxyethyl methacrylate)-treated 24-well tissue culture plates. 750 μL of supplemented Stem Pro-34 media was added 1 day after sorting. To generate monolayer cultures, 80 μL of the single cell suspension was spotted on individual wells of 25% Matrigel-coated 12-well tissue culture plates in supplemented Stem Pro-34 media. After 1 day, 1 mL of supplemented StemPro-34 media was added.

To isolate hematopoietic cells from OP9-DL4 co-cultures, human cells were isolated by Magnetic Activated Cell Sorting (MACS). The cultures were dissociated with Trypsin for 5 minutes at 37° C. and stained with the Mouse Cell Depletion Cocktail (1:5, Miltenyi) at 100,000,000 cells/mL for 15 minutes at 4° C. in the dark. Following MACS on an MS Column (Miltenyi), the flow-through fraction was stained with fluorescent-conjugated antibodies in Calcium and Magnesium-free PBS (CellGro) supplemented with 2% FCS and DNaseI for 30 minutes at 4° C. in the dark. Cells were sorted and collected in Calcium and Magnesium-free PBS supplemented with 2% FCS and DNaseI or deposited into wells of 96-well tissue culture plates containing 150 μL of supplemented culture media and stromal cells where indicated.

To isolate cells to generate macrophages, CD43⁺ cells from cultures on day 9 of differentiation were generally used. The large numbers of colony-forming progenitors in the day 9 cultures made them a useful starting population to differentiate macrophages. CD43⁺ cells were isolated by MACS. Single cell suspensions from the day 9 cultures were stained with CD43 MicroBeads (1:5, Miltenyi) at 100,000,000 cells/mL for 15 minutes at 4° C. in the dark. Following MACS on an MS Column (Miltenyi), the bound fraction was cryopreserved for future differentiation to the macrophage fate in CryoStor-10 (BioLife) freeze media at 2,000,000 cells per vial in 0.5 mL.

The following antibodies were used: KDR-PE (3:20, clone 89106, R&D Systems), KDR-Biotin (1:10, clone 89106, Novus Biologicals) CD235a/b-APC (1:100, clone HIR2/GA-R2, BD Pharmingen), CD34-PE-Cy7 (1:100, clone 4H11, ThermoFisher), CD34-APC (1:100, clone 8G12, BD Pharmingen), CD34-FITC (1:100, clone 8G12, BD Pharmingen), CD43-PE (3:100, clone 1G10, BD Pharmingen), CD43-FITC (1:10, clone 1G10, BD Pharmingen), CD43-APC-H7 (1:100, clone 1G10, BD Pharmingen), CD45-eFluor450 (1:50, clone HI30, ThermoFisher), CD45-APC-Cy7 (3:100, clone 2D1, BD Pharmingen), CD45-BV605 (1:50, clone HI30, BioLegend), CD117-APC (1:50, clone 104D2, BD Pharmingen), CD117-PE (1:50, clone 104D2, BD Pharmingen), CD144-APC (3:100, clone BV9, BioLegend), CD31-FITC (3:20, clone WM59, BD Pharmingen), CD31-PE (3:100, clone WM59, BD Pharmingen), CD90-APC (1:1000, clone 5E10, BD Pharmingen), CD7-PE (7:100, clone M-T701, BD Pharmingen), CD7-APC (3:100, clone 124-1D1, ThermoFisher), CD56-eFluor450 (1:50, clone TULY56, ThermoFisher), CD56-APC (3:100, clone B159, BD Pharmingen), CD5-FITC (1:10, clone UCHT2, BD Pharmingen), CD4-PE-Cy7 (1:50, clone SK3, ThermoFisher), CD8-PE (1:20, clone HIT8a, BD Pharmingen), CD3-FITC (1:50, clone UCHT1, ThermoFisher), TCRαβ-APC (3:100, clone IP26, BioLegend), TCRγδ-PE (5:100, clone IMMU510, Beckman Coulter), TCRγδ-BV421 (2:100, clone B1, BioLegend), CD19-PE-Cy7 (1:50, SJ25C1, BD Pharmingen), CD11b-APC (1:50, clone VIM12, Life Technologies), TCR Vγ9-APC (1:50, B3, BioLegend), TCR Vδ2-BV421 (1:50, B6, BioLegend), TCR Vδ1-APC (2:100, REA173, Miltenyi), CD68-PE-Cy7 (2:100, Y1/82A, BioLegend), CD14-PE (2:100, M5E2, BioLegend), CD163-BV421 (2:100, GHI/61, BioLegend) and CD64-APC-Cy7 (5:100, 10.1, BioLegend). Staining with biotin-conjugated antibodies was detected with Streptavidin-BV421 (1:100, BioLegend) or Streptavidin-PE-Cy7 (1:200, BD Pharmingen).

In experiments that examined aldehyde dehydrogenase activity, the Aldefluor assay was used (STEMCELL Technologies). Cells were incubated in the Aldefluor assay buffer containing BSA (0.1%) and BAAA substrate (0.12 μg/mL) for 60 minutes at 37° C. Thereafter, cells were washed in ice cold IMDM supplemented with FCS (2%), Aldefluor assay buffer (10%) and stained with antibodies against various cell surface markers, as described above. An aldehyde dehydrogenase inhibitor, DEAB (0.75 nM)-treated sample was used as a negative control.

Flow cytometry and cell sorting data were analyzed using FlowJo software (Tree Star).

### Quantitative Real-Time PCR

Total RNA was prepared with the RNAqueous RNA Isolation Kit (ThermoFisher) that included treatment with RNase-free DNase. Reverse transcribed to cDNA was performed using the iScript cDNA Synthesis Kit (Bio-Rad). RT-qPCR was performed on a CFX384 Touch Real-Time PCR Detection System (Bio-Rad) with the QuantiFast SYBR Green PCR Kit (Qiagen). Gene expression was evaluated as ΔCt relative to TBP. For globin analyses, gene expression was evaluated as ΔCt relative to ACTB. Genomic DNA content was assessed using primers for the GAGEB1 promoter. Primer sequences are listed in Table 1.1.

TABLE 1.1
Primer sequences for RT-qPCR.

| | Forward primer | Reverse primer |
|---|---|---|
| | sequence | sequence |
| Human gene | (5′-3′) | (5′-3′) |
| TBP | TGAGTTGCTCAT | CCCTCAAACCAA |
| (SEQ ID NO: | ACCGTGCTGCTA | CTTGTCAACAGC |
| 1, 2) | | |
| ACTB | AAACTGGAACGG | CAATGTGCAATC |
| (SEQ ID NO: | TGAAGGTGACAG | AAAGTCCTCGGC |
| 3, 4) | | |
| GAGEB1 | CAACATACCTCA | CCCTATGTTCCT |
| (SEQ ID NO: | TAGCATTATACA | GGTTCTTCATAT |
| 5,6) | AGAC | T |
| RUNX1a/b | CGTGCACATACA | CCTCCACGAATC |
| (SEQ ID NO: | TTAGTAGCACTA | TTGCTTGCAGAG |
| 7,8) | CC | GT |
| SCL | CCGTGGATTCGC | GAAAGAAGAGGG |
| (SEQ ID NO: | TTGAGTTA | AGCCAGAAG |
| 9, 10) | | |
| NOTCH1 | CGGGTCCACCAG | GTTGTATTGGTT |
| (SEQ ID NO: | TTTGAATG | CGGCACCAT |
| 11,12) | | |
| NOTCH2 | TTTGGCAACTAA | TGCCAAGAGCAT |
| (SEQ ID NO: | CGTAGAAACTCA | GAATACAGAGA |
| 13, 14) | AC | |
| NOTCH3 | CTCCTGCATCCT | GACTGAGAGGGT |
| (SEQ ID NO: | TGCCTTG | GGGTGGTA |
| 15, 16) | | |
| NOTCH4 | CATTAAAAGGCA | CGTGGAAGATGT |
| (SEQ ID NO: | GGCTGGAA | CTGCTCTG |
| 17,18) | | |
| HES1 | AGCTGGTGCTGA | CTACTGAGCAAG |
| (SEQ ID NO: | TAACAGCGGAAT | TGCTGAGGGTTT |
| 19, 20) | | |
| HES5 | TGGGTGCCTCCA | GCTTCCACGTGA |
| (SEQ ID NO: | CTATGATCCTTA | CTGAGAGTTCAA |
| 21, 22) | | |
| HEY1 | TGATCATGGTGT | GCAACCACAGTT |
| (SEQ ID NO: | GCGAGTGGTCAA | CCATGCACCAAA |
| 23, 24) | | |
| HEY2 | GAGTGAGAGAGT | ACTTCTGTCCCT |
| (SEQ ID NO: | CGTGTTTC | TTCCTTTC |
| 25, 26) | | |
| HBE | TCTGGCTACTCA | TCACAGGAACAC |
| (SEQ ID NO: | CTTTGGCAAGGA | CTGCAAACTGGA |
| 27, 28) | | |
| HBG1/2 | TGGGAAATGTGC | AAGCTCTGAATC |
| (SEQ ID NO: | TGGTGACCGTTT | ATGGGCAGTGAG |
| 29, 30) | | |
| HBB | ACTAAGCTCGCT | TCCAGATGCTCA |
| (SEQ ID NO: | TTCTTGCTGTCC | AGGCCCTTCATA |
| 31, 32) | | |

### Hemangioblast Colony-Forming Assay

Hemangioblast colony-forming potential was performed by plating 10,000 to 20,000 cells in 1% methylcellulose (1%, Shin-Etsu) supplemented with FCS (10%), D4T endothelial cell conditioned-medium (20%) (can be replaced with StemPro or similar media), L-glutamine (2 mM), ascorbic acid (25 μg/mL), transferrin (150 μg/mL), monothioglycerol (33 μg/mL), FGF2 (10 ng/mL), VEGF (10 ng/mL), IL6 (10 ng/mL), IL11 (5 ng/mL), SCF (100 ng/mL) and SB431542 (6 μM). Cultures were maintained in hypoxic conditions (37° C., 5% CO₂, 5% O₂) for 6 days prior to quantification.

### Hematopoietic Colony-Forming Assay

Colony-forming progenitor number was quantified by plating 100 to 80,000 cells (e.g. 100 cells from sorted populations derived from the day 6 CD34+CD43− cells that were differentiated for 5 additional days and up to 80,000 cells from day 15 of differentiation of unsorted cultures) in methylcellulose (1%) containing plasma-derived serum (15%, Animal Technologies), protein-free hybridoma media II (5%, Invitrogen), L-glutamine (2 mM), ascorbic acid (25 μg/mL), transferrin (150 μg/mL), monothioglycerol (33 μg/mL), TPO (50 ng/mL), IL3 (50 ng/mL), IL6 (10 ng/mL), IL11 (5 ng/mL), SCF (100 ng/mL), EPO (4 Units/mL), GM-CSF (1 ng/mL), M-CSF (10 ng/mL), IGF1 (25 ng/mL), VEGF (10 ng/mL) and FGF2 (10 ng/mL). Cultures were maintained in normoxic conditions (37° C., 5% CO₂) for 9 to 14 days prior to quantification.

### OP9-DL4 Co-Culture for T Lymphoid Differentiation

OP9 stromal cells that constitutively express Delta-like 4 (OP9-DL4) (Schmitt and Zuniga-Pflucker, 2002, 2006) were used to assay T lymphoid potential. Co-cultures were maintained in normoxic conditions (37° C., 5% CP₂). 1 to 250,000 cells were cultured with OP9-DL4 cells on 0.1% gelatin-treated tissue culture plates in αMEM (Gibco) supplemented with penicillin/streptomycin (1%), FCS (20%, HyClone), L-glutamine (2 mM), IL7 (5 ng/mL) and FLT3L (5 ng/mL). SCF (30 ng/mL) was included at the start of the co-culture and removed after 4-6 days. Cultures were transferred to new OP9-DL4 cells every 4-6 days by vigorous pipetting and passage through a 40 μm strainer. Cultures were analyzed by flow cytometry at the indicated stages and scored positive if greater than 10 CD45⁺CD56⁻CD4⁺CD8⁺ events were observed.

### HUVEC-E4ORF1 Co-Culture for Hematopoietic Cell Expansion

Human umbilical vein endothelial cells (HUVECs) that were engineered to express E4ORF1 (HUVEC-E4ORF1) (Seandel et al., 2008) were used to assay hematopoietic cell expansion. Co-cultures were maintained in normoxic conditions (37° C., 5% CO₂). 25 cells were cultured with HUVEC-E4ORF cells on Collagen type 1 (0.3 mg/mL, Fujifilm)-treated tissue culture plates in Stemspan SFEM (STEMCELL Technologies) supplemented with L-glutamine (2 mM), TPO (50 ng/mL), SCF (100 ng/mL), VEGF (10 ng/mL), FGF2 (10 ng/mL), FLT3L (20 ng/mL), IL3 (50 ng/mL), IL7 (20 ng/mL), IL6 (10 ng/mL). Cultures were analyzed by flow cytometry at the indicated stages.

### Hematopoietic Multipotent Progenitor Cell Assay

Hematopoietic cells were isolated from OP9-DL4 co-cultures initiated with day 6 CD34⁺CD43⁻ cells after 5 days. Single cells were sorted into the wells of 96-well plates retaining index sorting information on an Ariall-RITT cell sorter. The perimeter wells were excluded from the sort. The cells were cultured in α-MEM supplemented with FCS (20%), penicillin/streptomycin (1%, ThermoFisher), L-glutamine (2 mM, ThermoFisher) and SCF (30 ng/mL), IL7 (5 ng/mL) and FLT3L (5 ng/mL). The cultures were maintained in normoxic conditions (37° C., 5% CO₂). After 4 days, the cultures were dissociated with Trypsin and 30% of the culture was transferred to methylcellulose to evaluate erythro-myeloid potential as described above. The cultures were scored after 14 days and erythroid colonies were picked for β globin expression analysis by RT-qPCR as described above. As the OP9-DL4 cells express GFP, the colonies were visualized under fluorescence to confirm their human identity. The remaining fraction of the culture was seeded on new OP9-DL4 cells to evaluate NK and T cell potential as described above. Cultures were dissociated with trypsin and transferred to fresh OP9-DL4 biweekly for another 12 to 14 days. OP9-DL4 co-cultures were analyzed by flow cytometry. Greater than 10 CD45⁺-gated events (T: CD7⁺CD56⁻CD4⁺CD8⁺, NK: CD56⁺CD7⁺) were required to call a positive well.

### Hematopoietic Cell Xenotransplantation

Animal experiments were performed in accordance to the institution guidelines approved by the University Health Network Animal Care Committee. Post-natal day 3 NOD-Prkdc^{scid}II2rg^{tm1Wjl}/SzJ (NSG) mice were irradiated with 100 cGy, 1 day prior to intrahepatic transplantation with cryopreserved CD34⁺ cord blood (25,000 cells, STEMCELL Technologies) and hPSC-derived CD34⁺CD45⁺CD90⁺CD7⁻ cells (4,000-9,000 cells). Four weeks after transplantation, mice were euthanized and the femurs were flushed through a 40 μm filter in IMDM to collect the marrows. Prior to antibody staining, the cells were incubated with anti-mouse CD16/32 (1:20, clone 93, ThermoFisher) for 10 minutes at 4° C. To reliably identify human cells, two human-specific CD45 antibody clones were used.

### Quantification and Statistical Analyses

All data are presented as the mean±the standard error of the mean (s.e.m.). Statistical analyses were performed in GraphPad Prism 8 or the R statistical computing environment as indicated in the figure captions.

### Example 2

Previous studies have shown that sequential addition of BMP4, and FGF2 and either with low levels of Activin A or the Wnt inhibitor, IWP2 promotes the generation of KDR+CD235a+ (herein referred to as CD235a/b to be consistent with the specificity of the antibody clone that was used) mesoderm that gives rise to primitive hematopoietic cells and some lineages of an EMP-like program. However, this mesoderm did not show any capacity to generate T lymphoid cells (Sturgeon et al., 2014) suggesting that it did not possess the full spectrum of hematopoietic potential observed in the mouse yolk sac. These findings may indicate that hematopoietic development of the mouse and human are different or that the CD235a/b+ mesoderm in the previous study was not properly specified. The effect of concentration and combination treatment was assessed. Concentrations of Activin A (0 to 10 ng/mL) in the presence of BMP4 (10 ng/mL) and FGF2 (5 ng/mL) from day 1 of differentiation (FIG. 1a) were assessed. The frequency of CD235a/b+ cells increased in an Activin A dose-dependent manner and plateaued when its concentration exceeded 6 ng/mL. Although the majority of the cells did not express KDR at day 3 of differentiation (FIGS. 1b and 1c), they did upregulate its expression by day 4 of differentiation (e.g. after 3 days of concurrent treatment with Activin A, FGF2 and BMP4). Under these conditions, cells induced with 10 ng/mL of BMP4, 5 ng/mL of FGF2 and 6 ng/mL of Activin A consistently gave rise to cultures that contained greater than 30% of KDR⁺CD235a/b⁺ cells (FIGS. 1b and 1d).

The cultures induced with BMP4, FGF2 and 6 ng/mL of Activin A were assessed for hematopoietic potential. EBs were cultured in the presence of VEGF, FGF2 and hematopoietic cytokines (FIG. 2a) and analyzed the cultures by flow cytometry over 9 days at 3-day intervals for the emergence of hematopoietic cells marked by the expression of CD43 and CD45. Hematopoietic cells were present at day 6 of differentiation and increased in number over the next 9 days of culture to day 15 of differentiation (FIGS. 2b and 2c) indicative of the expansion and maturation of the primitive hematopoietic program. To formally determine whether the hematopoietic cells belonged to the primitive program, I assayed colony-forming progenitor potential in methylcellulose cultures. The number of colony-forming progenitors rapidly increased between days 6 and 9 of differentiation and then decreased over the next 6 days (FIG. 2d). Erythroid progenitors that generated small, compact colonies of erythroblasts dominated the cultures (FIGS. 2d and 2e). RT-qPCR revealed that the cells within these colonies predominantly expressed the embryonic β globin, HBE (FIG. 2f) confirming that they represent the human primitive erythroid lineage.

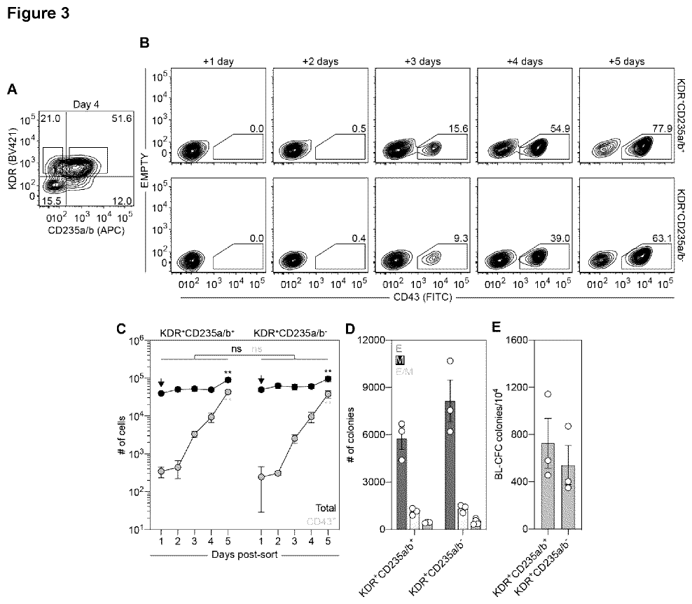

Further experiments were conducted to confirm that the primitive hematopoietic cells develop from KDR⁺CD235a/b⁺ mesoderm. CD235a/b⁺ and CD235a/b⁻ fractions of KDR⁺ population were isolated by FACS at day 4 of differentiation and cultured them in the presence of VEGF, FGF2 and hematopoietic cytokines for 5 days (FIG. 3a). The two KDR⁺ populations generated CD43⁺ hematopoietic cells that increased in number over 5 days of culture (FIGS. 3b and 3c). The populations generated comparable numbers of colony-forming progenitors, the majority of which gave rise to small colonies of primitive erythroblasts (FIG. 3d). Analyses of hemangioblast potential revealed that the frequency of BL-CFCs was equal between the two KDR⁺ populations (FIG. 3e). However, given the higher proportion of KDR⁺CD235a/b⁺ cells relative to KDR⁺CD235a/b⁻ cells, the majority of BL-CFCs are contained within the KDR⁺CD235a/b⁺ population. The presence of primitive erythroid progenitors and BL-CFCs in the two KDR populations demonstrate that both have primitive hematopoietic potential. This suggests that either primitive hematopoiesis is not restricted to the KDR⁺CD235a/b⁺ mesoderm or that the day 4 KDR⁺CD235a/b⁻ population developed from day 3 mesoderm that expressed CD235a/b. Collectively, these data demonstrate that KDR⁺CD235a/b⁺ mesoderm gives rise to the progenitors of the primitive program.

### The Primitive Program Transitions Through a Hemogenic Endothelial Cell Intermediate

The expression of CD43 was monitored by flow cytometry to define earliest stage that primitive hematopoietic cells appear. As shown in FIG. 4a, CD43⁺ cells were first observed on day 6 of differentiation. Analyses of the cultures 24 hours earlier, prior to the emergence of CD43⁺ cells, revealed the presence of a population that displayed the surface marker phenotype of HECs, including the expression of vascular markers, KDR, CD34, KIT, CD144 and CD31 and a lack of the hematopoietic markers, CD43 or CD45 (FIGS. 4b and 4c). RT-qPCR analyses also showed that two transcription factors associated with HECs, RUNX1 and SCL/TAL1 were expressed in the KDR⁺CD34⁺ population (FIG. 4d) further supporting the interpretation that this population contains HECs that give rise to the hematopoietic cells of the primitive program.

The KDR⁺CD34⁺ population was isolated on day 5 of differentiation by FACS and the cells were cultured as aggregates in the presence of VEGF, FGF2 and hematopoietic cytokines for 7 days (FIG. 5a). A small population of CD43⁺ hematopoietic cells after 1 day that became increasingly abundant over the duration of the culture (FIGS. 5b and 5c). Analyses of colony-forming progenitor potential in methylcellulose showed transient progenitor activity within the aggregates that predominantly gave rise to small erythroid colonies characteristic of the primitive program (FIG. 5d). To provide additional evidence that the primitive program transitions through a HEC intermediate, day 5 KDR⁺CD34⁺ population was plated on Matrigel to visualize endothelial-to-hematopoietic transition (EHT). An adherent monolayer formed within 1 day of culture that rapidly gave rise to a population of round, non-adherent CD43⁺ hematopoietic cells (FIGS. 5e and 5f).

### The Primitive Program is Dependent on NOTCH Signaling

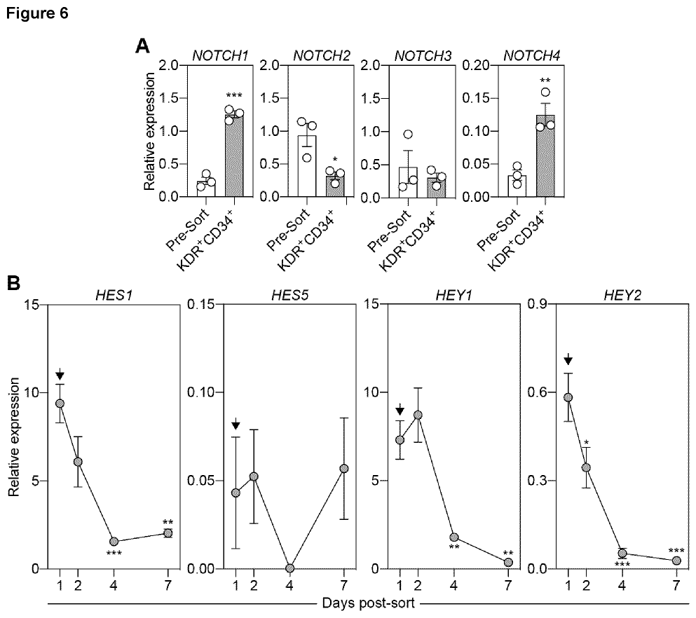

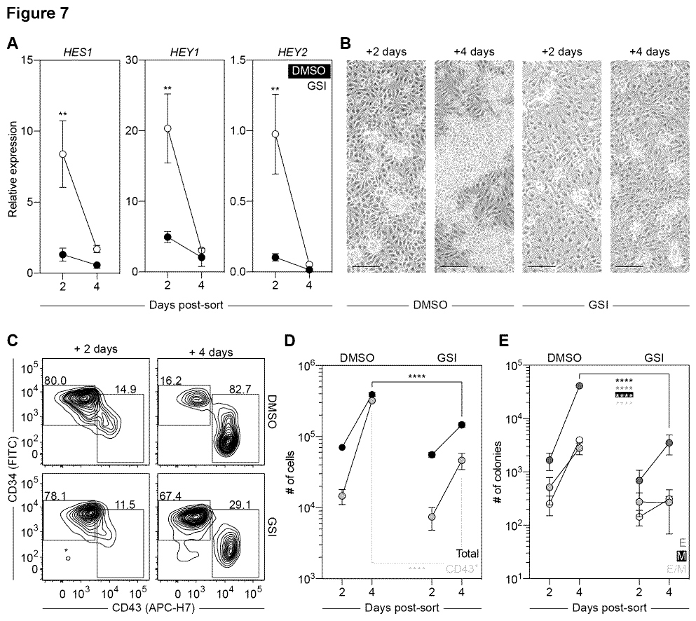

Studies of mouse embryonic hematopoiesis have demonstrated that Notch signaling is dispensable for the development of the primitive and EMP programs, but essential for the generation of HSCs (Hadland et al., 2004). The expression of the NOTCH receptors (NOTCH1, NOTCH2, NOTCH3, NOTCH4) was analyzed and found that NOTCH1 and NOTCH4 were expressed in the KDR+CD34+ population at day 5 of differentiation (FIG. 6a). To determine if this pathway is required for primitive hematopoiesis isolated KDR+CD34 + cells were cultured as aggregates in the presence of VEGF, FGF2 and hematopoietic cytokines. RT-qPCR analyses confirmed this finding and showed elevated levels of expression of the NOTCH target genes, HES1, HEY1 and HEY2 during the first 2 days of culture suggesting that the emergence of the primitive program is regulated by NOTCH. The expression levels declined over the next 5 days of culture, coincident with the generation and expansion of primitive hematopoietic cells (FIG. 6b)., Day 5 KDR+CD34+ population were also cultured in the presence of gamma-secretase inhibitor (GSI) to inhibit NOTCH signaling. GSI treatment reduced the expression of NOTCH target genes (HES1, HEY1 and HEY2; FIG. 7a) confirming the inhibition of the pathway. In contrast to mouse models, the number of CD43+ hematopoietic cells and colony-forming progenitors were reduced in the presence of GSI (FIGS. 7b to 7d) strongly suggesting that NOTCH signaling is required for the generation of the human primitive program.

### Identification of a Second Hemogenic Endothelial Cell Population with Broader Hematopoietic Lineage Potential

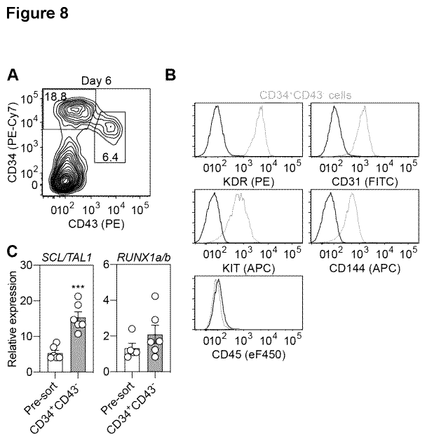

In addition to the emergent CD43+ hematopoietic cells on day 6 of differentiation, the EBs also contained a CD34+CD43−CD45− population that co-expressed the HEC/endothelial cell markers KDR, KIT, CD144 and CD31 (FIGS. 8a and 8b). RT-qPCR analyses also showed that the CD34+CD43− population expressed the HEC transcription factors RUNX1 and SCL/TAL1 (FIG. 8c) suggesting the presence of HECs.

To characterize the hematopoietic potential of the day 6 CD43+ and CD34+CD43− populations, the populations were isolated by FACS and the cells cultured as aggregates in the presence of VEGF, FGF2 and hematopoietic cytokines for 6 days (FIG. 9a). The CD43+ population expanded over the initial 3 days of culture and was stable thereafter (FIGS. 9b and 9c). Although the number of hematopoietic cells progressively increased over the course of the culture, the number of colony-forming progenitors decreased between days 3 to 6 of culture (FIG. 9d) suggestive of the exhaustion of the primitive program. In contrast, cultures generated from the CD34+CD43− population gave rise to a small CD43+ population following 1 day of culture that increased in size over the 6 days of culture (FIGS. 9b and 9c). The total number of colony-forming progenitors also increased over this time (FIG. 9d). Analyses of colony subtypes showed a dominance of the erythroid lineage at all stages in the CD43+-derived population. In contrast, the CD34+CD43−-derived population showed a more balanced distribution of erythroid and myeloid progenitors between days 1 and 3 of culture. The erythroid lineage dominated at 6 days of culture (FIG. 9e). Analyses of colony morphologies showed that the CD43+ population largely gives rise to small colonies of erythroblasts. In contrast, colony-forming cells that gave rise to large erythroid colonies were abundant in populations derived from the CD34+CD43− cells between days 1 and 3 of culture. Colony-forming progenitors that generated small erythroid colonies dominated after 6 days of culture (FIG. 9f) and likely originated from the progenitors that gave rise to large colonies at earlier stages of culture.

CD45 is expressed on the hematopoietic progenitors of the EMP, LMP and definitive programs, but not those of the primitive program in mouse (Ferkowicz et al., 2003), Expression of CD45 on cells generated from the day 6 CD43+ and CD34+CD43− populations was monitored. Cultures derived from the CD43+ population contained a small CD45+ population following 6 days of culture, which likely represent the maturing myeloid cells of the primitive program. In contrast, cultures generated from the CD34+CD43− population contained a large number of CD45+ cells that may represent emerging EMPs (FIGS. 10a to 10c). To test this, the following fractions derived from the CD34+CD43− population were isolated by FACS after 5 days of culture: CD34+CD45+KIT+, CD34+CD45+KIT−, CD34−CD45+and CD34−CD45− cells and assayed them for colony-forming progenitor potential (FIG. 10d). KIT was included as it is expressed on mouse EMPs (Ferkowicz et al., 2003; McGrath et al., 2015; Mikkola et al., 2003). The CD34−CD45− population was highly enriched in small erythroid colony-forming progenitors that likely represent a combination of contaminating primitive erythroid progenitors and EMP-derived erythroid progenitors that have matured to the stage at which they have downregulated CD45. The majority of the progenitors in the CD45+ populations were of the mast cell and macrophage lineages (FIGS. 10e and 10f). Granulocyte progenitors were also detected in these cultures. Erythroid lineage cells were also generated by this population, however they were largely associated with mixed erythro-myeloid colonies. RT-qPCR of these colonies showed higher levels of expression of the fetal β globin, HBG relative to colonies generated from the day 6 CD43+ population (FIG. 10g) indicating that the CD34+CD45+KIT+-derived erythroid cells are representative of the human EMP lineage. Taken together, these findings identify the emergence of distinct populations of CD34+ cells that display developmental potential indicative of the human primitive and EMP programs.

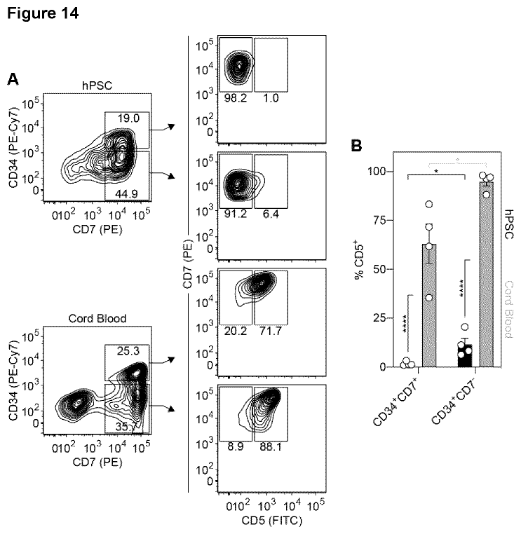

Given the emergence of a program with EMP-like potential, the CD34+CD43− population was assessed to see if it also contained T lymphoid progenitors. Both the day 6 CD34+CD43− and CD43+ populations were cultured with OP9-DL4 cells that have been engineered to provide levels of NOTCH signaling required for human T cell development (Mohtashami et al., 2013). As shown in FIG. 11a, the CD34+CD43−, but not CD43+ population generated CD45+CD56−CD4+CD8+ T lymphoid progenitors following 1 month of culture with OP9-DL4 cells. αβ or γδ TCR+ lymphocytes were detected following an additional 10 days of culture (FIGS. 11b and 11c). To confirm that the T lymphoid lineage was generated from the KDR+CD235a/b+ yolk sac-like mesoderm, this population was isolated and cultured as aggregates for 3 days and then seeded them onto OP9-DL4 cells (FIGS. 12a and 12b). During the 3 day aggregation culture, the KDR+CD235a/b+ cells generated a population that consisted of approximately 85% CD34+CD43− cells. Analyses after 1 month of culture revealed the presence of CD4+CD8+ T lymphoid progenitors demonstrating that KDR+CD235a/b+ mesoderm does give rise to the T lymphoid lineage (FIG. 12c). To determine if this T lymphoid lineage is different from that generated from cord blood hematopoietic progenitors, the expression of TCR genes was compared in mature CD3+ lymphocytes. CD3+ lymphocytes were detected in hPSC- and cord blood-derived cultures from day 25 and their frequency increased over the next 15 days of culture (FIGS. 13a and 13b). of the CD3+ cells, γδ T lymphocytes dominated the TCR+ population at early stages of culture. as the cultures progressed, αβ T lymphocytes became the major TCR+ population in both cultures (FIGS. 13c and 13d). in the human embryo, γδ lymphocytes that express the tcrdv gene 2 (Vδ2) are abundant at early developmental stages. this lineage gives way to Vδ1 lymphocytes later in development (Dimova et al., 2015; Haynes and Heinly, 1995; Haynes et al., 1988; McVay and Carding, 1996) possibly corresponding to the transition from yolk sac t lymphopoiesis to those generated from hscs. Consistent with this interpretation, flow cytometric analyses of Vδ1 and Vδ2 expression in CD3+ lymphocytes showed that Vδ2 was the dominant TCRDV receptor express on the hpsc-derived T cells, while the Vδ1 expressing T cells were the predominant population generated from cord blood cells (FIGS. 13e to 13g). To determine whether differences in T lymphoid development are apparent at earlier stages of culture, the surface marker expression of CD7+ progenitors was analyzed after 12 days of culture, as this represents one of the earliest stages of lymphoid commitment. Analyses of the cord blood-derived cells showed that CD5 was expressed on the differentiating CD7+CD34+ T lymphoid progenitor population. In contrast, the hPSC-derived CD7+CD34+ cells were CD5− (FIG. 14). Collectively, these findings show that KDR+CD235a/b+ mesoderm induced with BMP4, FGF2 and Activin A contains primitive, EMP and T lymphoid potential, largely reflecting the hematopoietic potential of the mouse yolk sac. These studies also provide evidence that the hPSC-derived T lymphocytes may differ from those generated from definitive cord blood progenitors.

### The Erythro-Myeloid and T Lymphoid Progenitors are Generated from Multipotent Hematopoietic Progenitors

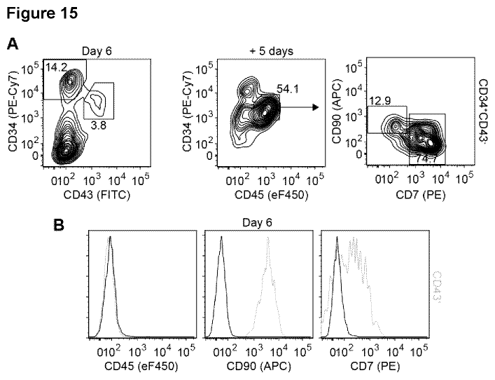

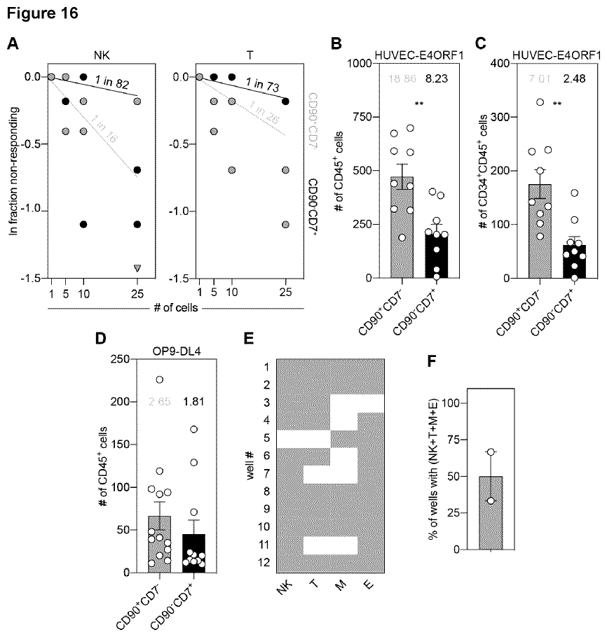

The close temporal association between the EMP and LMP programs in the mouse and the demonstration that the hPSC-derived CD34+CD43− HEC population contained erythroid, myeloid and lymphoid potentials raised the possibility that these lineages develop from a common multipotent progenitor. To search for this progenitor, day 6 CD34+CD43− cells were cultured with OP9-DL4 cells for 5 days to initiate EHT and then analyzed the developing hematopoietic populations for the presence of cells that express markers of hematopoietic progenitors, including CD34, CD45, CD90 and CD7. From these analyses, distinct CD34+CD45+CD90+CD7− and CD34+CD45+CD90−CD7+ populations were identified (FIG. 15a). Although CD90 and CD7 were expressed in day 6 CD43+ primitive hematopoietic population, these cells did not express CD45 (FIG. 15b). Limiting dilution analyses to determine if the frequency of lymphoid progenitors in either the CD90+CD7− or CD90−CD7+CD34+CD45+ subpopulations was high enough to carry out single cell clonal analyses. These studies showed that the frequencies of NK (CD90+CD7−: 1:16 and CD90−CD7+: 1:82) and T lymphoid (CD90+CD7−: 1:26 and CD90−CD7+: 1:73) progenitors were higher in the CD90+CD7+ population (FIG. 16a). The proliferative potential of the two CD34+CD45+ subpopulations was measured by culturing the cells with HUVEC-E4ORF1 endothelial cells, which have been shown to support the expansion of hematopoietic progenitors (Seandel et al., 2008). Consistent with its higher hematopoietic progenitor frequency, the CD90+CD7− population gave rise to significantly more total CD45+ and more CD34+CD45+ cells than the CD90−CD7+ population after 4 days of co-culture (FIGS. 16b and 16c). These data suggested that the CD90+CD7− population is suitable target population for clonal analyses and that HUVEC-E4ORF1 endothelial cells are able to efficiently expand hematopoietic progenitors. Although the HUVEC-E4ORF1 cells supported hematopoietic expansion, NK cell and T lymphoid potentials were lost from both CD34+CD45+ subpopulations following 4 days of culture (data not shown). By comparison, culture with OP9-DL4 cells for 8 days produced fewer CD45+ hematopoietic cells (FIG. 16d), but allowed for progenitors with lymphoid potential to be maintained. To determine if short-term culture with OP9-DL4 cells is supportive of multilineage hematopoiesis, 25 CD34+CD45+CD90+CD7− cells were cultured on OP9-DL4 stroma for 4 days then assayed 30% of the culture for erythroid and myeloid progenitors in methylcellulose and the remaining cells for lymphoid potential by co-culture with fresh OP9-DL4 cells. Of the 12 wells tested, 6 (50%) gave rise to lymphoid, erythroid and myeloid progeny (FIGS. 16e and 16f). These data demonstrate that co-culture with OP9-DL4 cells for 4 days preserves the multilineage hematopoietic potential of the CD34⁺CD45⁺CD90⁺CD7⁻ population.

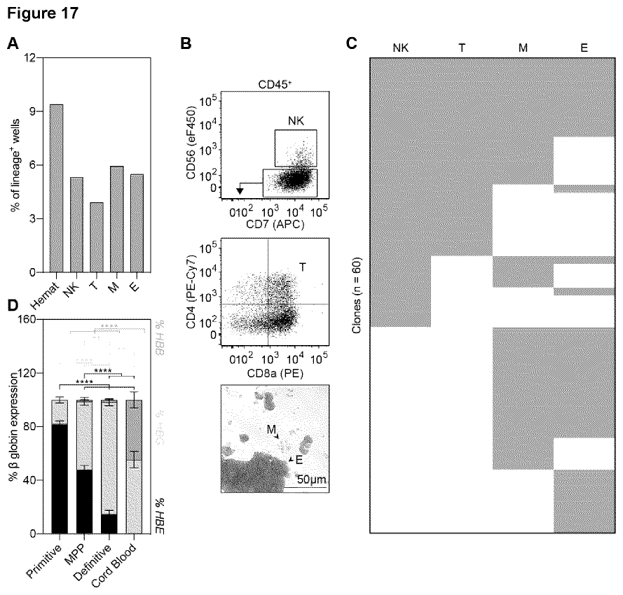

To determine whether the CD34⁺CD45⁺CD90⁺CD7⁻ population contains multipotent hematopoietic progenitors, single cells were deposited on OP9-DL4 cells by FACS. After 4 days, the cultures were harvested and assayed for erythroid, myeloid and lymphoid potentials, as described above. From 637 sorted CD34⁺CD45⁺CD90⁺CD7⁻ cells, 60 cells (9.4%) produced a hematopoietic clone (FIG. 17a). Of the 60 clones, 10 (16.7%) contained NK cell, T lymphoid, erythroid and myeloid progeny (FIGS. 17b and 17c) demonstrating that multipotent hematopoietic progenitors are contained within the CD34⁺CD45⁺CD90⁺CD7⁻ population. The remaining 50 clones were largely restricted to the lymphoid (20%) or erythro-myeloid (25%) fates. β globin expression of the erythroid colonies generated from each clone was compared to erythroid colonies derived from the primitive and definitive programs. The erythroid colonies generated from the CD34⁺CD45⁺CD90⁺CD7⁻ progenitors expressed lower levels of the embryonic β globin, HBE and higher levels of the fetal β globin, HBG than colonies of primitive erythroid cells and higher levels of HBE and lower levels of HBG than colonies generated from hPSC-derived definitive progenitors (FIG. 17d). This intermediate pattern is consistent with the β globin expression pattern observed in the EMP-derived erythroid cells in the mouse. Taken together, these data demonstrate that the day 6 CD34⁺CD43⁻ HECs give rise to multipotent hematopoietic progenitors and in doing so, define a human multilineage yolk sac program that displays erythroid, myeloid and lymphoid potential. Given these findings, the EMP and LMP terminology used to describe the hematopoietic programs in the mouse yolk sac can be revised to the multipotent progenitor program (MPP) for the human yolk sac.

### Engraftment Potential of Multipotent Hematopoietic Progenitors

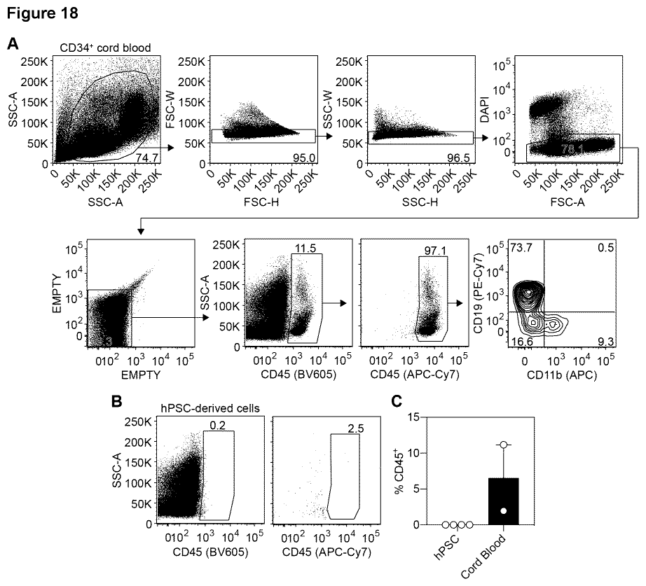

Given the presence of multipotent hematopoietic progenitors in the CD34⁺CD45⁺CD90⁺CD7⁻ population, the population was assessed to see if it contains engraftable progenitors similar to those identified in the E9.5 mouse yolk sac (Yoder and Hiatt, 1997; Yoder et al., 1997a; Yoder et al., 1997b). To test this, between 4,000 and 9,000 hPSC-derived CD34⁺CD45⁺CD90⁺CD7⁻ cells were transplanted into the livers of irradiated NOD.Cg-Prkdc^{scid}II2rg^{tm1Wjl}/SzJ (NSG) neonatal mice. Based on the clonal analyses, the transplanted populations contained between 375 and 850 hematopoietic progenitors, of which 62 to 140 cells were multipotent. CD34⁺ cord blood cells were transplanted as controls. Analyses of the bone marrow of recipients 4 weeks following transplantation showed engraftment only in animals that received CD34⁺ cord blood cells (FIG. 18).

### Aldehyde Dehydrogenase Activity During Hematopoietic Development

Studies of mouse hematopoietic development have demonstrated that RA signaling mediated by Raldh2 is required for the specification of HSCs and that AGM-derived HSCs can be isolated based on this activity using the Aldefluor assay (Chanda et al., 2013). Studies in the human have also shown that cord blood HSCs can be isolated based on Aldefluor activity (Fallon et al., 2003; Hess et al., 2004). To determine whether RA signaling could play a role in the specification and development of the human yolk sac programs, ALDHs were measured with the Aldefluor assay in CD34⁺CD45⁺CD90⁺CD7⁻ cells, as well as in mesoderm (days 3 and 4 of differentiation) and HEC-containing populations (days 5 and 6 of differentiation) from which they were generated. Aldefluor is a molecule that fluoresces and is generated from a non-fluorescent precursor in a reaction catalyzed by aldehyde dehydrogenases (including, but not limited to RALDH2).

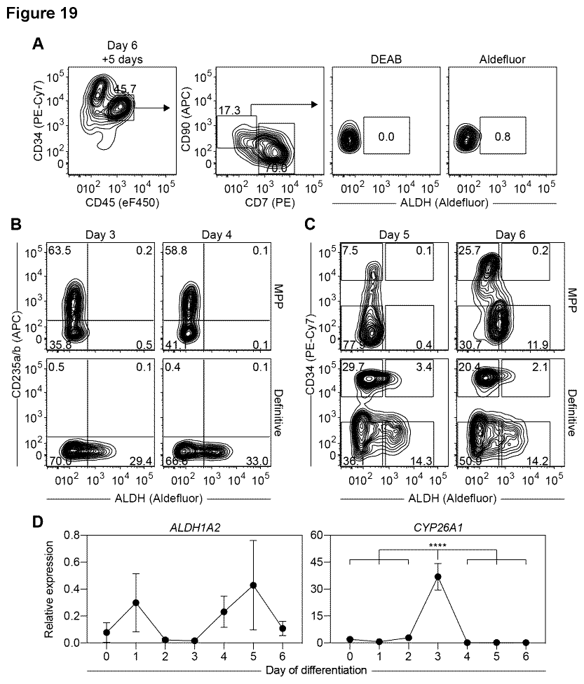

As shown in FIGS. 19a to 19c, most of the populations did not contain ALDH⁺ cells. While ALDH⁺ cells were detected on day 6 of differentiation, they were restricted to the CD34− fraction, which likely represents a non-hematopoietic population (FIG. 19c). In contrast, populations specified to the definitive fate contained readily detectable ALDH⁺ populations across days 3 to 6 of differentiation (FIGS. 19b and 19c). The expression of ALDH1A2, which encodes RALDH2, between days 0 and 6 of differentiation was also analyzed. It was found that ALDH1A2 is consistently expressed at low levels across these stages of culture (FIG. 19d), presumably too low to generate sufficient enzymatic activity to be detected by the Aldefluor assay. In contrast to the pattern of ALDH1A2, expression of CYP26A1, the enzyme responsible for the degradation of RA, showed a sharp peak at day 3 of differentiation (FIG. 19d). Taken together, these findings suggest that RA signaling does not play a role in the generation of the human primitive or MPP programs. Rather, the high levels of expression of CYP26A1 at day 3 of differentiation suggests that CD235a/b⁺ mesoderm is safeguarded from exogenous RA signaling.

### Hematopoietic Development from Human Pluripotent Stem Cell Lines

To determine whether the protocol for the generation of the human yolk sac hematopoietic programs is broadly applicable to other hPSC lines, iPSC line (CHOP10WT) generated from the peripheral blood of a healthy donor (Maguire et al., 2016) was tested. hPSC lines can differ in their responsiveness to cytokines, so Activin A (0 to 10 ng/mL) concentration in the presence of BMP4 (10 ng/mL) and FGF2 (5 ng/mL) between days 1 and 4 of differentiation was titrated to optimize the induction of KDR⁺CD235a/b⁺ mesoderm. Populations induced with either 2 or 4 ng/mL of Activin A generated the highest proportion of KDR⁺CD235a/b⁺ cells at day 4 of differentiation (FIGS. 20a and 20b). The mesoderm induced with 2 ng/mL of Activin A was differentiated in the presence of VEGF, FGF2 and hematopoietic cytokines to evaluate the hematopoietic potential of the population. CD43⁺ hematopoietic cells appeared on day 6 of differentiation and increased in number over an additional 6 days of differentiation (FIGS. 20c and 20d) consistent with the pattern observed in previous studies using H1 hESCs (FIGS. 2b and 2c). These findings indicate that the induction of KDR⁺CD235a/b⁺ mesoderm in different hPSC lines require different concentrations of Activin A and highlights the importance of optimizing this stage of the protocol for each line.

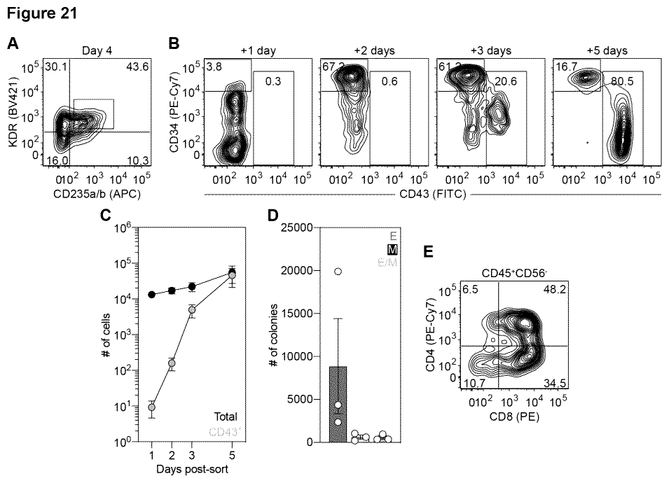

KDR⁺CD235a/b⁺ mesoderm generated from the CHOP10WT iPSCs was assessed for its ability to give rise to the primitive program and T lymphoid lineages. KDR⁺CD235a/b⁺ mesoderm cells were isolated by FACS and cultured the cells as aggregates in the presence of VEGF, FGF2 and hematopoietic cytokines (FIG. 21a). A CD34⁺CD43⁻ population appeared after 1 day of culture and preceded the emergence of CD43⁺ hematopoietic cells, which were generated over the subsequent stages of the culture (FIGS. 21b and 21c). Analyses of colony-forming potential after 5 days of culture confirmed that KDR⁺CD235a/b⁺ mesoderm gives rise to the primitive program (FIG. 21d). T lymphoid progenitors were observed following the culture of KDR⁺CD235a/b⁺-derived cells with OP9-DL4 cells for 1 month (FIG. 21e). Taken together, these data demonstrate that it is possible to generate KDR⁺CD235a/b⁺ mesoderm with primitive erythroid and T lymphoid potential from different hPSC lines using the BMP4, FGF2 and Activin A-based protocol described herein.

The development of the human yolk sac hematopoietic programs is described herein. These lineages are generated from KDR⁺CD235a/b⁺ mesoderm that was induced with specific concentrations of BMP4, FGF2 and Activin A. Within 24 hours, this mesoderm generates primitive HECs that quickly undergo NOTCH-dependent EHT to produce hematopoietic progenitors. These cells give rise to primitive erythroid, mast cell and macrophage progeny. As primitive hematopoietic progenitors emerge, a second population of HECs is present. These HECs undergo EHT to give rise to multipotent hematopoietic progenitors that can be distinguished from primitive progenitors based on the expression of CD45. These MPPs show EMP erythroid, mast cell, macrophage, granulocyte, NK cell and T lymphoid potential consolidating the EMP and LMP programs. A schematic of the model is provided in FIG. 22.

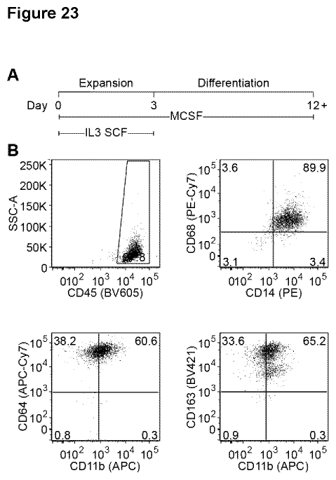

Given the therapeutic potential of these lineages, a protocol to generate cultures that are highly enriched in macrophages has been developed. In this protocol, hematopoietic progenitors are expanded and committed to the macrophage fate in media containing SCF, IL3 and MCSF for 3 days. Thereafter, the SCF and IL3 are withdrawn from the culture allowing for the maturation of the macrophage progenitors in the culture (FIG. 23a). After 13-15 days, macrophages defined by the expression of CD45, CD68, CD11b, CD14, CD64 and CD163 were the predominant lineage in the culture (FIG. 23b).

### Example 3

### Producing T Lineage Cell Differentiation

iPSCs are treated with a PSC culture composition comprising a BMP receptor agonist (BMPRA) and optionally a ROCK inhibitor (Ri) for about 1 day to produce a BMPRA-Ri population of cell. The BMPRA-Ri population of cells is then treated with a mesoderm specifying culture composition comprising a BMPR1/R2 agonist, an FGF receptor agonist and an activin receptor agonist for about 3 additional days (day 4 of differentiation) to produce KDR+CD235a/b+ mesoderm cells.

In some embodiments, the KDR+CD235a/b+ mesoderm cells are cultured for at least 2 days in for example VEGF, FGF2, IL-6 and IL-11 to produce day 6 CD34⁺CD43⁻ HECs. In yet some other embodiments the day 6 CD34⁺CD43⁻ HECs are further cultured for about 5 days in SCF, IL-7 and FLT3L with a Notch ligand, optionally a scaffold comprising and immobilized Notch ligand such as DL4 or OP9-DL4 cells, to obtain day 6+5 CD34⁺CD45⁺ progenitors.

To generate populations of T cells, the cells described above, were co cultured with stromal cells that constitutively express Delta-like 4 (OP9-DL4). Co-cultures are maintained in normoxic conditions (37° C., 5% CO₂). The cells described above, (day 4 KDR⁺ mesoderm, day 6 CD34⁺CD43⁻ HECs or day 6+5 CD34⁺CD45⁻ progenitors) are cultured with for example on OP9-DL4 cells on a gelatin-treated tissue culture plate in a suitable medium such as in αMEM (Gibco) supplemented with antibiotics, FCS (20%, HyClone), L-glutamine (2 mM), IL7 (5 ng/mL) and FLT3L (5 ng/mL). SCF (30 ng/mL) can be included at the start of the co-culture and removed after 4-6 days. Cultures are transferred to new OP9-DL4 cells every 4-6 days by vigorous pipetting and passage through a 40 μm strainer for 3-4 weeks. Desired T cells are identified using established lineage markers, including CD45, CD3, and one of pan-TCRαβ, pan-TCRγδ or Vδ2. For example, to isolate Vδ2 T cells, the combination of antibodies directed to CD45, CD3 and Vδ2 can be used. The antibodies can be conjugated to magnetic particles and magnetically isolated. Alternatively the cells can be isolated by FACS (FLUORSCENCE ACTIVATED CELL SORTING).

### REFERENCES

Abud, E. M., Ramirez, R. N., Martinez, E. S., Healy, L. M., Nguyen, C. H. H., Newman, S. A., Yeromin, A. V., Scarfone, V. M., Marsh, S. E., Fimbres, C., et al. (2017). iPSC-Derived Human Microglia-like Cells to Study Neurological Diseases. Neuron 94, 278-293.e279.
Chanda, B., Ditadi, A., Iscove, N. N., and Keller, G. (2013). Retinoic acid signaling is essential for embryonic hematopoietic stem cell development. Cell 155, 215-227.
Dimova, T., Brouwer, M., Gosselin, F., Tassignon, J., Leo, O., Donner, C., Marchant, A., and Vermijlen, D. (2015). Effector Vgamma9Vdelta2 T cells dominate the human fetal gammadelta T-cell repertoire. Proc Natl Acad Sci U S A 112, E556-565.
Fallon, P., Gentry, T., Balber, A.E., Boulware, D., Janssen, W. E., Smilee, R., Storms, R. W., and Smith, C. (2003). Mobilized peripheral blood SSCloALDHbr cells have the phenotypic and functional properties of primitive haematopoietic cells and their number correlates with engraftment following autologous transplantation. Br J Haematol 122, 99-108.
Ferkowicz, M. J., Starr, M., Xie, X., Li, W., Johnson, S. A., Shelley, W. C., Morrison, P. R., and Yoder, M.C. (2003). CD41 expression defines the onset of primitive and definitive hematopoiesis in the murine embryo. Development 130, 4393-4403.
Haideri, S. S., McKinnon, A. C., Taylor, A. H., Kirkwood, P., Starkey Lewis, P. J., O'Duibhir, E., Vernay, B., Forbes, S., and Forrester, L. M. (2017). Injection of embryonic stem cell derived macrophages ameliorates fibrosis in a murine model of liver injury. NPJ Regen Med 2, 14.
Haynes, B. F., and Heinly, C. S. (1995). Early human T cell development: analysis of the human thymus at the time of initial entry of hematopoietic stem cells into the fetal thymic microenvironment. J Exp Med 181, 1445-1458.
Haynes, B. F., Singer, K. H., Denning, S. M., and Martin, M. E. (1988). Analysis of expression of CD2, CD3, and T cell antigen receptor molecules during early human fetal thymic development. J Immunol 141, 3776-3784.
Hess, D. A., Meyerrose, T. E., Wirthlin, L., Craft, T. P., Herrbrich, P.E., Creer, M. H., and Nolta, J. A. (2004). Functional characterization of highly purified human hematopoietic repopulating cells isolated according to aldehyde dehydrogenase activity. Blood 104, 1648-1655.
Klichinsky, M., Ruella, M., Shestova, O., Lu, X. M., Best, A., Zeeman, M., Schmierer, M., Gabrusiewicz, K., Anderson, N. R., Petty, N. E., et al. (2020). Human chimeric antigen receptor macrophages for cancer immunotherapy. Nature Biotechnology 38, 947-953.
Liu, E., Marin, D., Banerjee, P., Macapinlac, H. A., Thompson, P., Basar, R., Nassif Kerbauy, L., Overman, B., Thall, P., Kaplan, M., et al. (2020). Use of CAR-Transduced Natural Killer Cells in CD19-Positive Lymphoid Tumors. New England Journal of Medicine 382, 545-553.
Maguire, J. A., Gagne, A. L., Jobaliya, C. D., Gandre-Babbe, S., Gadue, P., and French, D. L. (2016). Generation of human control iPS cell line CHOPWT10 from healthy adult peripheral blood mononuclear cells. Stem Cell Res 16, 338-341.
McGrath, K. E., Frame, J. M., Fegan, K. H., Bowen, J. R., Conway, S. J., Catherman, S. C., Kingsley, P. D., Koniski, A. D., and Palis, J. (2015). Distinct Sources of Hematopoietic Progenitors Emerge before HSCs and Provide Functional Blood Cells in the Mammalian Embryo. Cell Rep 11, 1892-1904.
McVay, L. D., and Carding, S. R. (1996). Extrathymic origin of human gamma delta T cells during fetal development. J Immunol 157, 2873-2882.
Mikkola, H. K., Fujiwara, Y., Schlaeger, T. M., Traver, D., and Orkin, S. H. (2003). Expression of CD41 marks the initiation of definitive hematopoiesis in the mouse embryo. Blood 101, 508-516.
Moroni, F., Dwyer, B. J., Graham, C., Pass, C., Bailey, L., Ritchie, L., Mitchell, D., Glover, A., Laurie, A., Doig, S., et al. (2019). Safety profile of autologous macrophage therapy for liver cirrhosis. Nature Medicine 25, 1560-1565.
Schmitt, T. M., and Zuniga-Pflucker, J. C. (2002). Induction of T cell development from hematopoietic progenitor cells by delta-like-1 in vitro. Immunity 17, 749-756.
Schmitt, T. M., and Zuniga-Pflucker, J. C. (2006). T-cell development, doing it in a dish. Immunol Rev 209, 95-102.
Seandel, M., Butler, J. M., Kobayashi, H., Hooper, A. T., White, I. A., Zhang, F., Vertes, E. L., Kobayashi, M., Zhang, Y., Shmelkov, S. V., et al. (2008). Generation of a functional and durable vascular niche by the adenoviral E4ORF1 gene. Proc Natl Acad Sci U S A 105, 19288-19293.
Sturgeon, C. M., Ditadi, A., Awong, G., Kennedy, M., and Keller, G. (2014). Wnt signaling controls the specification of definitive and primitive hematopoiesis from human pluripotent stem cells. Nat Biotechnol 32, 554-561.
Trotman-Grant, A. C., Mohtashami, M., De Sousa Casal, J. et al. DL4-μbeads induce T cell lineage differentiation from stem cells in a stromal cell-free system. Nat Commun 12, 5023 (2021). https://doi.org/10.1038/s41467-021-25245-8
Yoder, M. C., and Hiatt, K. (1997). Engraftment of embryonic hematopoietic cells in conditioned newborn recipients. Blood 89, 2176-2183.
Yoder, M. C., Hiatt, K., Dutt, P., Mukherjee, P., Bodine, D. M., and Orlic, D. (1997a). Characterization of definitive lymphohematopoietic stem cells in the day 9 murine yolk sac. Immunity 7, 335-344.
Yoder, M. C., Hiatt, K., and Mukherjee, P. (1997b). In vivo repopulating hematopoietic stem cells are present in the murine yolk sac at day 9.0 postcoitus. Proc Natl Acad Sci U S A 94, 6776-6780.

1. A method of producing a KDR+CD235a/b+ mesoderm cells capable of giving rise to T lymphoid lineage cells or cells differentiated therefrom, the method comprising:
contacting pluripotent stem cells (PSCs) with a PSC culture composition comprising a BMP receptor agonist (BMPRA) and optionally a ROCK inhibitor (Ri) to produce a BMPRA-Ri population of cells;
contacting the BMPRA-Ri population of cells with a mesoderm specifying culture composition comprising a BMPR1/R2 agonist, an FGF receptor agonist and an activin receptor agonist to produce KDR+CD235a/b+ mesoderm cells.

2. The method of claim 1, wherein the PSCs are contacted with the mesoderm specifying culture composition for about 3 days, at least 3 days or up to 3 days; wherein the pluripotent stem cells and/or the BMPRA-Ri population of cells are in the form of embryoid bodies; and/or wherein the pluripotent stem cells are embryonic stem cells or induced pluripotent stem cells, optionally human induced pluripotent stem cells.

3.-4. (canceled)

5. The method of claim 1, wherein the BMPRA and/or BMPR1/R2 agonist is BMP4; wherein the FGF receptor agonist is or comprises FGF2 and/or wherein the activin receptor agonist is activin A.

6.-8. (canceled)

9. The method of claim 1, wherein the method further comprises contacting the KDR+CD235a/b+ mesoderm cells with a HEC culture composition comprising VEGF and optionally FGF2 or one or more hematopoietic cytokines to obtain CD34+KDR+ hemogenic endothelial cells (HECs).

10. The method of claim 9, further comprising culturing the CD34+KDR+ hemogenic endothelial cells (HECs) in a primitive progenitor culture composition to obtain CD43+ hematopoietic progenitor cells or culturing the CD34+KDR+ HECs for a period of time and isolating CD34+CD43− HECs, optionally for about 1 day.

11. The method of claim 10, wherein the method further comprises culturing the CD43+ hematopoietic progenitor cells in the primitive progenitor culture composition to obtain primitive program lineage cells, optionally wherein the one or more of the primitive program lineage cells are isolated;
wherein the method further comprises culturing the CD43+ hematopoietic progenitor cells with a macrophage permissive cocktail and isolating macrophage cells;
wherein the method further comprises culturing the CD43+ hematopoietic progenitor cells with a mast cell permissive cocktail and isolating mast cells; or
wherein the method further comprises culturing the CD43+ hematopoietic progenitor cells with an erythroid cell permissive cocktail and isolating primitive erythrocytes.

12.-15. (canceled)

16. The method of claim 1, wherein the ROCK inhibitor is Y-27632 and/or wherein the BMPRA is BMP4.

17.-19. (canceled)

20. The method of claim 10, wherein the method further comprises contacting the CD34+CD43− HECs with a multipotent progenitor culture composition comprising a Notch agonist to obtain CD34+CD45+, optionally CD34+CD45+CD90+CD7− and/or CD34+CD45+CD90−CD7+ hematopoietic progenitor cells, and optionally expanding the CD34+CD45+ hematopoietic progenitor cells.

21. (canceled)

22. The method of claim 20, wherein the method further comprises contacting the CD34+CD45+ hematopoietic progenitor cells or the expanded CD34+CD45+ hematopoietic progenitor cells to obtain multipotent lineage cells optionally wherein one or more of the multipotent lineage cells are isolated.

23. (canceled)

24. The method of claim 22, wherein macrophage cells are isolated, mast cells are isolated, erythrocytes are isolated, granulocytes are isolated or T lymphocytes are isolated, optionally wherein the isolated T lymphocytes are gamma/delta, alpha/beta, T lymphocytes, optionally Vdelta2 T lymphocytes.

25.-28. (canceled)

29. The method of claim 20, wherein the Notch agonist is a Notch ligand, optionally provided via a Notch ligand-conjugated tissue culture plate or bead.

30. The method of claim 1, wherein one or more types of the isolated cells are resuspended in a composition optionally wherein the composition comprises a gel or is a sterile osmotically balanced fluid solution and/or wherein the composition comprises one or more other types of cells.

31.-32. (canceled)

33. A population of cells or composition comprising one or more types of the isolated cells of claim 1, wherein the composition comprises a carrier, a gel, and/or an osmotically balanced fluid solution.

34.-36. (canceled)

37. The population of cells or composition of claim 33, wherein the composition is sterile, or wherein the population of cells or composition comprises cardiomyocytes or hepatocytes.

38. A cell implant comprising a gel or a scaffold, and one or more types of isolated cells prepared according to the method of claim 1, or a population of cells or composition comprising said one or more types of isolated cells.

39. A mesoderm specifying culture additive comprising:
a BMPR1/R2 agonist,
an FGF receptor agonist and
an activin receptor agonist;
a mesoderm specifying culture composition comprising a hematopoetic progenitor suitable base media and the mesoderm specifying culture additive; or
a kit comprising the mesoderm specifying culture additive or the culture composition.

40. The mesoderm specifying culture additive or culture composition or kit of claim 39, wherein the BMPR1/R2 agonist is BMP4, the FGF receptor agonist is FGF2, and/or the activin receptor agonist is Activin A.

41.-42. (canceled)

43. The mesoderm specifying culture additive or culture composition or kit of claim 40, wherein the amount of: the BMP4 is sufficient to provide within 0.5 ng/mL to about 100 ng/mL, the FGF2 is sufficient to provide within 0.5 ng/mL-100 ng/mL and the Activin A is sufficient to provide within 0.5 ng/mL-100 ng/ml, in a solution of about 500 mL.

44.-63. (canceled)

64. The mesoderm specifying culture additive or culture composition or kit of claim 43, wherein the ratio of BMP4 to FGF2 to Activin A is about 10:5:6 or about 10:5:2 or within about 10:5:6 to about 10:5:2.

65.-66. (canceled)

67. The cell implant of claim 38, wherein the scaffold is a pouch.


SEQUENCE LISTING (32 sequences)

SEQ ID NO: 1 - DNA, 24 bp, Homo Sapiens
tgagttgctcataccgtgctgcta

SEQ ID NO: 2 - DNA, 24 bp, Homo Sapiens
ccctcaaaccaacttgtcaacagc

SEQ ID NO: 3 - DNA, 24 bp, Homo Sapiens
aaactggaacggtgaaggtgacag

SEQ ID NO: 4 - DNA, 24 bp, Homo Sapiens
caatgtgcaatcaaagtcctcggc

SEQ ID NO: 5 - DNA, 28 bp, Homo Sapiens
caacatacctcatagcattatacaagac

SEQ ID NO: 6 - DNA, 25 bp, Homo Sapiens
ccctatgttcctggttcttcatatt

SEQ ID NO: 7 - DNA, 26 bp, Homo Sapiens
cgtgcacatacattagtagcactacc

SEQ ID NO: 8 - DNA, 26 bp, Homo Sapiens
cctccacgaatcttgcttgcagaggt

SEQ ID NO: 9 - DNA, 20 bp, Homo Sapiens
ccgtggattcgcttgagtta

SEQ ID NO: 10 - DNA, 21 bp, Homo Sapiens
gaaagaagagggagccagaag

SEQ ID NO: 11 - DNA, 20 bp, Homo Sapiens
cgggtccaccagtttgaatg

SEQ ID NO: 12 - DNA, 21 bp, Homo Sapiens
gttgtattggttcggcaccat

SEQ ID NO: 13 - DNA, 26 bp, Homo Sapiens
tttggcaactaacgtagaaactcaac

SEQ ID NO: 14 - DNA, 23 bp, Homo Sapiens
tgccaagagcatgaatacagaga

SEQ ID NO: 15 - DNA, 19 bp, Homo Sapiens
ctcctgcatccttgccttg

SEQ ID NO: 16 - DNA, 20 bp, Homo Sapiens
gactgagagggtgggtggta

SEQ ID NO: 17 - DNA, 20 bp, Homo Sapiens
cattaaaaggcaggctggaa

SEQ ID NO: 18 - DNA, 20 bp, Homo Sapiens
cgtggaagatgtctgctctg

SEQ ID NO: 19 - DNA, 24 bp, Homo Sapiens
agctggtgctgataacagcggaat

SEQ ID NO: 20 - DNA, 24 bp, Homo Sapiens
ctactgagcaagtgctgagggttt

SEQ ID NO: 21 - DNA, 24 bp, Homo Sapiens
tgggtgcctccactatgatcctta

SEQ ID NO: 22 - DNA, 24 bp, Homo Sapiens
gcttccacgtgactgagagttcaa

SEQ ID NO: 23 - DNA, 24 bp, Homo Sapiens
tgatcatggtgtgcgagtggtcaa

SEQ ID NO: 24 - DNA, 24 bp, Homo Sapiens
gcaaccacagttccatgcaccaaa

SEQ ID NO: 25 - DNA, 20 bp, Homo Sapiens
gagtgagagagtcgtgtttc

SEQ ID NO: 26 - DNA, 20 bp, Homo Sapiens
acttctgtccctttcctttc

SEQ ID NO: 27 - DNA, 24 bp, Homo Sapiens
tctggctactcactttggcaagga

SEQ ID NO: 28 - DNA, 24 bp, Homo Sapiens
tcacaggaacacctgcaaactgga

SEQ ID NO: 29 - DNA, 24 bp, Homo Sapiens
tgggaaatgtgctggtgaccgttt

SEQ ID NO: 30 - DNA, 24 bp, Homo Sapiens
aagctctgaatcatgggcagtgag

SEQ ID NO: 31 - DNA, 24 bp, Homo Sapiens
actaagctcgctttcttgctgtcc

SEQ ID NO: 32 - DNA, 24 bp, Homo Sapiens
tccagatgctcaaggcccttcata